(19) United States
(12) Patent Application Publication
(10) Pub. No.: US 2025/0155422 A1
(43) Pub. Date: May 15, 2025

(54) INLINE WATER QUALITY MONITORING DEVICE FOR BATHING UNIT SYSTEM AND METHOD OF MAINTAINING SAME

(71) Applicant: GECKO ALLIANCE GROUP INC., Québec (CA)

(72) Inventors: Guillaume Tremblay, Québec (CA); Vincent Bouchard, L’Ancienne-Lorette (CA)

(21) Appl. No.: 18/505,707

(22) Filed: Nov. 9, 2023

(51) Int. Cl. G01N 33/18

(52) U.S. Cl. CPC G01N 33/1893

(57) ABSTRACT

A water quality monitoring device is provided for use with a bathing unit system. The monitoring device includes a housing configured to be fluidly coupled with a circulation system of the bathing unit system and comprising an upper portion and a lower portion which forms a chamber, wherein the housing is to allow water from the receptacle to flow through the chamber. The lower portion is releasably engaged with the upper portion and forms a container for holding fluid when disengaged from the upper portion. The monitoring device further includes a probe coupled to the upper portion of the housing and extending into the chamber, wherein the probe is configured to measure water quality measurements of the water flowing though the chamber and wherein, when the lower portion is disengaged from the upper portion, the probe is exposed and remains in situ relative to the upper portion.

### TECHNICAL FIELD

The present invention relates generally to the monitoring of water quality in bodies of water, and more specifically, to a method, device and system for monitoring quality measurements in bodies of water, including for example monitoring oxidation-reduction potential (ORP) levels, pH levels, temperature and/or turbidity in a body of water such as in bathing units, including, but not limited to, a swimming pool, a spa, a hot tub, and other sports, recreational and therapeutic bodies of water.

### BACKGROUND

A bathing unit system, such as for example a spa, a hot tub or pool, typically includes various components such as a receptacle holding water, one or more pumps to circulate water in a circulation system comprising a plurality of conduits, a heating module to heat the water, a filter system, an air blower, a lighting system, and a control system for activating and managing the various components.

To maintain the water of a bathing unit system at sanitary conditions, the bathing unit system may include a variety of sanitizing and filtering components. For example, the filter system may remove foreign material, such as hair, soil, or solids, from the water. In addition, the bathing unit system also require regular addition of sanitization agents in order to maintain sanitary conditions. Typical sanitation agents include halogen chemicals, in particular free chlorine and bromine. Such conventional halogen-based sanitization agents often require that the concentration of halogen chemicals be maintained within a specified range, which is typically between approximately 3 parts per million (ppm) and approximately 5 ppm. Allowing levels of the sanitization agent to either fall below or rise above required levels may result in decreased efficiency of the bathing unit system. For example, low levels of sanitization agents in the bathing unit system can contribute to algae blooms, bacterial breakouts, cloudiness in the water, and chemical imbalances. If left untreated, water-borne bacteria can afflict users of the bathing units with a variety of health problems and illnesses, such as pseudomonas, rashes, hot tub lung, car infections, etc. On the other hand, high levels of sanitization agents in the bathing unit system can also be harmful to users of the bathing unit system, and may cause rashes and chemical burns, and may further be harmful to components of the bathing unit system and may cause deterioration of plastic-based components and/or corrosion of metal-based components.

However, maintaining a suitable concentration of sanitization agents in the bathing unit typically requires the user to perform periodic measurements by, e.g., using water testing kits and then taking action to adjust the concentration of the sanitation agents so that it lies within the specified range. Such actions may involve, e.g., the user adding water to reduce the concentration of the sanitization agents and/or causing an increase the concentration of the sanitation agents. Manually measuring and maintaining the concentration of sanitization agents is often a lengthy process which is not always diligently followed by the user, often resulting in less-than-ideal water conditions.

In practice, the quality and chemical composition of the water of the bathing unit system may be affected by one or more of environmental factors and weather conditions (e.g., rain, sunlight, hail, wind, pollen, debris, and the like) and human elements (e.g., bacteria, urine, and sweat). In order to account for these factors, conditions and elements, a user may also be required to perform periodic adjustments to the chemical composition of the water on a weekly (or more frequent basis). Such periodic adjustments may involve adding the sanitization agents as described above, but may also involve an addition of chemicals to maintain other chemical properties, such as pH, etc. Manually measuring and maintaining such chemical composition of the water may also be a manually intensive and time-consuming process, which is not always diligently followed by a user and may be susceptible to human error, while third party services can be expensive.

To address such deficiencies, various devices for measuring water quality measurements of water in a circulation system of a bathing unit system, such as pH, water temperature, oxidation-reduction potential (ORP), and turbidity have been proposed.

In some known systems, a water quality monitoring device may be provided in a housing designed to float on the surface of the water in the receptacle of the bathing unit system. These floating systems suffer from numerous drawbacks. For example, a floating system may interfere with swimmers or others in the water. Further, the user may need to enter the swimming pool or spa to access the floating system to read the quality measurements.

In other known systems, a water quality monitoring device is provided in a housing configured to be installed inline within a water circulation path of the bathing unit system so that water from the receptacle of the bathing unit system which enters the water circulation path flows through the water quality monitoring device. Such systems typically include a probe that contacts the water as it flows through the water quality monitoring device. The probe measures water quality measurements and typically include: (i) a pH sensor; (ii) a temperature sensor; and (iii) an ORP sensor. For example, patent publication nos. US 2022/0089459 and US 2020/0271635, the contents of which are incorporated by reference in their entirety, describe such inline monitoring devices.

However, a deficiency of conventional inline water quality monitoring devices is that, in order to prolong the useful life of the probe used to measure the water quality measurements, maintenance involving physically removing the probe from the housing needs to be performed on a periodic basis.

For example, in order to clean the probe, the probe may need to be physically removed from the inline water quality monitoring device to expose the end of the probe used to measure water quality measurements. Such a requirement increases the likelihood that the probe will be damaged through handling by a user inadvertently dropping the probe on the ground after it has been removed, for example. Additionally, when the bathing unit system is turned off for an extended period of time (e.g., during months in winter or other non-use periods), it is often advisable to remove the probe from the inline water quality monitoring device and store it in a separate enclosure in order to prolong its useful life. The separate enclosure typically needs to be filled with water or other storage fluid so that the end of the probe used to measure the water quality measurements remains wet. Such handling and storage requirements are an added burden for the user and may again also increase the likelihood that the probe will be damaged through handling. Further still, conventional inline water quality monitoring devices typically do not provide suitable mechanisms for easily calibrating (or recalibrating) the probe. Such calibrating (and recalibrating) is needed since, over time, the pH readings and/or ORP readings have a tendency to shift so that the readings progressively loose accuracy.

Against the background described above, there is a need in the industry to provide an inline water quality monitoring device and methods of maintaining such a device (e.g., cleaning, storing and/or calibrating, etc.) that alleviate at least in part the problems associated with existing water quality monitoring devices.

### SUMMARY

A problem sought to be addressed is to provide an inline water quality monitoring device and one or more methods of maintaining such an inline water quality monitoring device.

In accordance with a first aspect, there is provided an inline water quality monitoring device for use with a bathing unit system, the bathing unit system including a receptacle for holding water and a circulation system for removing water from and returning water to the receptacle. The inline water quality monitoring device comprises: a) a housing comprising an upper portion and a lower portion which forms a chamber, wherein the housing is configured to be fluidly coupled with the circulation system to allow water from the receptacle to flow through the chamber, and wherein the lower portion is releasably engaged with the upper portion and forms a container for holding fluid when disengaged from the upper portion; and b) a probe coupled to the upper portion of the housing and extending into the chamber, wherein the probe is configured to measure water quality measurements of the water from the receptacle which flows though the chamber and wherein, when the lower portion is disengaged from the upper portion, the probe is exposed and remains in situ relative to the upper portion.

The lower portion may be releasably engaged with the upper portion by being at least one of threadedly releasably engaged with the upper portion, magnetically releasably engaged with the upper portion, frictionally releasably engaged with the upper portion, or rotationally releasably engaged with the upper portion.

The lower portion may comprise a grip component on an outer surface of the lower portion. In a specific practical implementation, the grip component may comprise a plurality of vane grips outwardly extending from the outer surface of the lower portion.

The container formed by the lower portion may have a U-shaped cross-section or a V-shaped cross-section.

In some specific practical implementations, the container formed by the lower portion may be transparent or translucent.

When used in a calibration mode, the container may hold calibration fluid for calibrating the probe. The calibration fluid may comprise a fluid having known quality measurements to calibrate the quality measurements measured by the probe. The lower portion with the calibration fluid held in the container may be configured to be releasably re-engaged with the upper portion while the probe remains in situ relative to the upper portion such that the probe contacts the calibration fluid held in the container.

When used in a storage mode, the container may hold storage fluid for storing the probe. The storage fluid may comprise a fluid having a chemical composition that reduces degradation of the probe. In a specific practical implementation, the storage fluid may comprise a potassium chloride (KCl) solution, although other suitable storage solution may be used. The lower portion with the storage fluid held in the container may be configured to be releasably re-engaged with the upper portion while the probe remains in situ relative to the upper portion such that the probe contacts the storage fluid held in the container.

When used in a cleaning mode, the lower portion may be disengaged from the upper portion to allow a user to access the probe while the probe is exposed and remains in situ relative to the upper portion.

The lower portion may be a first lower portion defining a first container, and the first lower portion may be configured to be disengaged from the upper portion to allow a second lower portion defining a second container different from the first container to be releasably engaged with the upper portion. The first container defined by the first lower portion may have a first volume and the second container defined by the second lower portion has a second volume smaller than the first volume.

The second container may hold calibration fluid for calibrating the probe when the inline water quality monitoring device is used in a calibration mode. Alternatively, the second container may hold storage fluid for storing the probe when the inline water quality monitoring device is used in a storage mode.

The housing may comprise an inlet port and an outlet port to fluidly couple the housing with the circulation system. The chamber may be positioned between the inlet port and the outlet port such that at least part of the water from the receptacle enters the chamber through the inlet port and exits the chamber when the through the outlet port.

The inlet port may extend upwardly from the chamber and the outlet port may extend downwardly from the chamber. Alternatively, the inlet port and the outlet port may both extend upwardly from the chamber, or both extend downwardly from the chamber. As a further alternative, the inlet port may extend downwardly from the chamber and the outlet port may extend upwardly from the chamber. As a still further alternative, the inlet port and the outlet port may both extend laterally from the chamber. In such embodiments the inlet port and the outlet port may both extend in a substantially similar lateral direction; or the inlet port and outlet port may extend in opposing lateral directions.

The inlet port may comprise at least one inlet valve configured to open and close the inlet port. The outlet port may comprise at least one outlet valve configured to open and close the outlet port.

The at least one inlet valve and the at least one outlet valve may be configured to be: a) locked in a closed position, thereby preventing water flow from the circulation system from entering into the chamber, when the inline water quality monitoring device is used in at least one of a calibration mode, storage mode or a cleaning mode; or b) locked in an open position, thereby permitting water flow from the circulation system to flow through the chamber, when the inline water quality monitoring device is used in a monitoring mode.

The inline water quality monitoring device may further comprise a support component extending from the upper portion of the housing, wherein the support component may be configured to fixedly couple the upper portion to a supporting structure of the bathing unit system such that, when the lower portion is disengaged from the upper portion, the probe remains in situ relative to the bathing unit system.

The probe may comprise at least one of a pH sensor, an oxidation-reduction sensor (ORP sensor), and a temperature sensor.

In accordance with another aspect, there is provided a method for in situ maintenance of an inline water quality monitoring device. The inline water quality monitoring device comprises A) a housing comprising an upper portion and a lower portion defining a chamber, wherein the housing is configured to be fluidly coupled with the circulation system to allow water from the receptacle to flow through the chamber, and wherein the lower portion is releasably engaged with the upper portion and forms a container for holding fluid when disengaged from the upper portion and B) a probe extending into the chamber through the upper portion and configured to measure water quality measurements of the water from the receptacle which flows though the chamber. The method comprises: a) ceasing flow of the water from the receptacle through the chamber of the inline water quality monitoring device; b) disengaging the lower portion from the upper portion to expose the probe; and c) performing at least one maintenance operation on the probe while the probe remains in situ relative to the upper portion.

The lower portion may comprise a grip component on an outer surface of the lower portion. Disengaging the lower portion from the upper portion may comprise: a) disengaging the lower portion from the upper portion by manipulating the lower portion via the grip component.

Performing the at least one maintenance operation may comprise operating the inline water quality monitoring device in one of: a) a calibration mode; b) a cleaning mode; or c) a storage mode.

In a specific implementation, operating the inline water quality monitoring device in the calibration mode may comprise: a) pouring a calibration fluid having known quality measurements into the container formed by the lower portion; and b) re-engaging the lower portion with the upper portion while the probe remains in situ relative to the upper portion such that the probe contacts the calibration fluid held in the container.

In some specific implementations, operating the inline water quality monitoring device in the calibration mode may further comprise: a) using the probe to obtain quality measurements corresponding to the calibration fluid held in the container g; b) transmitting the obtained quality measurements to at least one processor associated with the bathing unit system; and c) performing, with the at least one processor, a computer-implemented calibration procedure including processing the quality measurements obtained by the probe and the known quality measurements of the calibration fluid.

In a specific practical implementation, at least part of the computer-implemented calibration procedure may be performed on a cloud-based server physically separate from the bathing unit system.

In a specific implementation, operating the inline water quality monitoring device in the storage mode may comprise: a) pouring storage fluid having a chemical composition which reduces degradation of the probe into the container formed by the lower portion; and b) re-engaging the lower portion with the upper portion while the probe remains in situ relative to the upper portion such that the probe contacts the storage fluid held by the container.

In a specific implementation, operating the inline water quality monitoring device in the cleaning mode may comprise: a) cleaning the probe while the probe is exposed and remains in situ relative to the upper portion; b) re-engaging the lower portion with the upper portion; and c) reinitiating the flow of the water from the receptacle to the container such that the cleaned probe contacts the water from the receptacle flowing through the chamber. Cleaning the probe may involve brushing the probe.

The housing may comprise an inlet port and an outlet port to fluidly couple the housing with the circulation system. The chamber may be positioned between the inlet port and the outlet port such that at least part of the water from the receptacle enters the chamber through the inlet port and exits the container through the outlet port. Ceasing flow of the water from the receptacle into the container may comprise: a) closing at least one inlet valve associated with the inlet port; and b) closing at least one outlet valve associated with the outlet port. In some implementations, the method may further comprise: a) locking the at least one inlet valve and the at least one outlet valve in a closed position when the inline water quality monitoring device is used in at least one of a calibration mode, a storage mode or a cleaning mode; and b) locking the at least one inlet valve and the at least one outlet valve in an open position when the inline water quality monitoring device is used in a monitoring mode.

In some implementations, the lower portion may be a first lower portion defining a first container having a first volume and performing the at least one maintenance operation may comprise: a) releasably engaging a second lower portion with the upper portion while the probe remains in situ relative to the upper portion, wherein the second portion defines a second container having a second volume different from the first volume.

The method may further comprise: a) pouring at least one of a calibration fluid or a storage fluid into the second container defined by the second lower portion prior to releasably engaging the second lower portion with the upper portion.

The method may further comprise: a) fixedly coupling the upper portion of the housing to a supporting structure of the bathing unit system such that performing the at least one maintenance operation on the probe while the probe remains in situ relative to the upper portion comprises performing the at least one maintenance operation on the probe while the probe remains in situ relative to the bathing unit system.

In accordance with another aspect, there is provided an inline water quality monitoring device for use with a bathing unit system, the bathing unit system including a receptacle for holding water and a circulation system for removing water from and returning water to the receptacle. The inline water quality monitoring device comprises: a) a housing comprising an upper portion and a lower portion which forms a chamber, wherein the housing is configured to be fluidly coupled with the circulation system to allow water from the receptacle to flow through the chamber, and wherein the lower portion is releasably engaged with the upper portion and forms a container for holding fluid when disengaged from the upper portion; and b) a probe coupled to the housing and extending into the chamber, wherein the probe is configured to obtain quality measurements of the water from the receptacle which flows though the chamber and wherein, when the lower portion is disengaged from the upper portion, the probe remains in situ relative to the housing.

The probe may be coupled to the upper portion of the housing and, when the lower portion is disengaged from the upper portion, the probe: a) may be exposed; and b) may remain in situ relative to the upper portion.

The probe may be coupled to the lower portion of the housing and, when the lower portion is disengaged from the upper portion, the probe: a) may extend into the container; and b) may remain in situ relative to the lower portion.

In accordance with another aspect, there is provided a method for in situ maintenance of an inline water quality monitoring device. The inline water quality monitoring device comprises A) a housing comprising an upper portion and a lower portion defining a chamber, wherein the housing is configured to be fluidly coupled with a circulation system of a bathing unit system to allow water from a receptacle of the bathing unit system to flow through the chamber, and wherein the lower portion is releasably engaged with the upper portion and forms a container for holding fluid when disengaged from the upper portion and B) a probe coupled to the housing and extending into the chamber and configured to measure water quality measurements of the water from the receptacle which flows though the chamber. The method comprises: a) ceasing flow of the water from the receptacle through the chamber of the inline water quality monitoring device; b) disengaging the lower portion from the upper portion; and c) performing at least one maintenance operation on the probe while the probe remains in situ relative to housing.

The probe may be coupled to the upper portion of the housing. Disengaging the lower portion from the upper portion may expose the probe while the probe remains in situ relative to the upper portion. Performing the at least one maintenance operation on the probe may comprise performing at least one maintenance operation on the probe while the probe is exposed and remains in situ relative to the upper portion.

The probe may be coupled to the lower portion of the housing and extends into the container. The probe may extend into the container when the lower portion is disengaged from the upper portion. Performing the at least one maintenance operation on the probe may comprise performing at least one maintenance operation on the probe while the probe extends into the container and remains in situ relative to the lower portion.

All features of embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Drawings.

### BRIEF DESCRIPTION OF THE DRAWINGS

A detailed description of the embodiments of the present invention is provided herein below, by way of example only, with reference to the accompanying drawings, in which:

FIG. 1 is a diagram of a bathing unit system incorporating a water quality monitoring device in accordance with one embodiment;

FIG. 2 is a perspective view of the water quality monitoring device of FIG. 1 in accordance with one embodiment;

FIG. 3 is a schematic view of the water quality monitoring device of FIG. 1 in accordance with an alternative embodiment;

FIGS. 4A-4C are schematic views of the water quality monitoring device of FIG. 1 coupled to an inlet conduit of a circulation forward path and an outlet conduit of a circulation return path in accordance with different embodiments;

FIG. 5A is a cross-sectional view of the water quality monitoring device of FIG. 1 with a lower portion shown engaged with an upper portion to form a chamber in accordance with one embodiment;

FIG. 5B cross-sectional view of the water quality monitoring device of FIG. 5A with the lower portion shown disengaged from the upper portion and forming a container in accordance with one embodiment;

FIG. 5C is a bottom plan view of the water quality monitoring device of FIG. 5A showing grip components in accordance with one embodiment;

FIGS. 6A-6D are cross-sectional views of the water quality monitoring device of FIG. 1 showing alternative embodiments of the lower portion shown engaged with the upper portion and disengaged from the upper portion;

FIGS. 7A and 7B are bottom plan views of the water quality monitoring device of FIG. 1 showing alternative embodiments of the grip components;

FIG. 8 is a flowchart of a method for operating the water quality monitoring device of FIG. 1 in a water quality monitoring mode in accordance with one embodiment; and

FIGS. 9A and 9B are flowcharts of a method for operating the water quality monitoring device of FIG. 1 in at least one maintenance mode in accordance with one embodiment.

It will be noted that throughout the appended drawings, like features are identified by like reference numerals. In the drawings, the embodiments of the invention are illustrated by way of examples. It is to be expressly understood that the description and drawings are only for the purpose of illustration and are an aid for understanding. They are not intended to be a definition of the limits of the present invention.

### DETAILED DESCRIPTION

The description below is directed to specific implementations and uses of embodiments of the invention in the context of bathing units. the term “bathing unit”, as used for the purposes of the present description, refers to spas/swim-spas, whirlpools, hot tubs, bath tubs, therapeutic baths and swimming pools and any other type of unit having a receptacle for holding water. Moreover, it is to be appreciated that while specific embodiments of the present invention have been described for use in the context of bathing units, the person skilled in the art will appreciate in view of the present description that alterative embodiments may be configured for use in an environment including a body of water other than a bathing unit in which measurement of water quality measurements may be of interest.

### Bathing Unit System 100

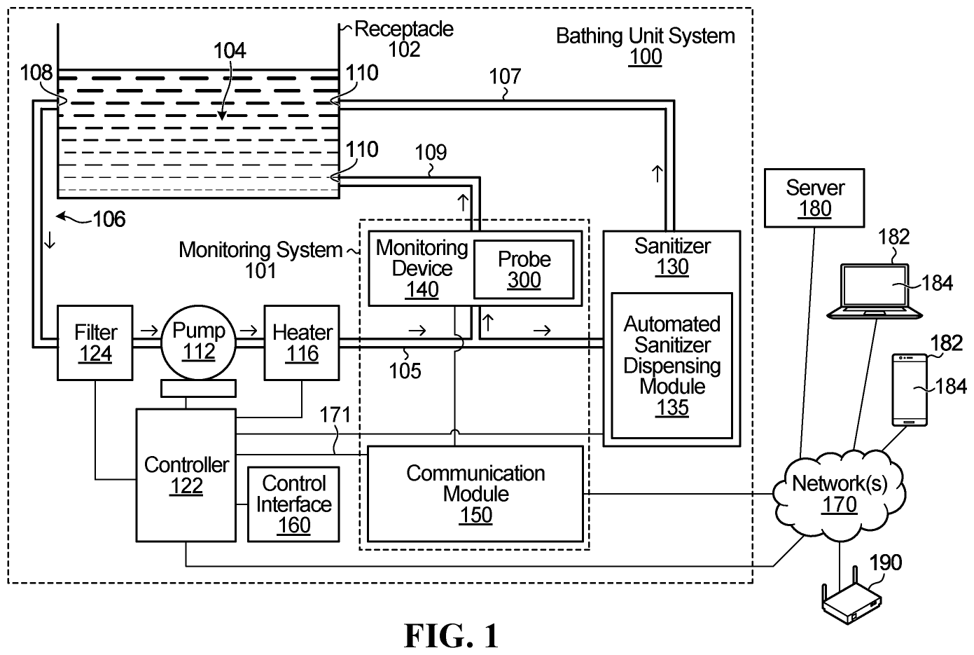

One embodiment of a bathing unit system 100 incorporating a water quality monitoring system 101 is shown in FIG. 1. In the embodiment shown, the bathing unit system 100 includes a receptacle 102 for holding water 104, a plurality of water inlets 110 (only two are shown) of the receptacle 102 which will typically be connected to respective jets, a plurality of water outlets 108 (only one is shown) of the receptacle 102, and a circulation system 106 including flow conduits for removing water from and returning water to the receptacle 102 through the water inlets 110 and the water outlets 108. The embodiment of the circulation system 106 shown in FIG. 1 has a primary circulation forward path 105, a primary circulation return path 107 and a secondary circulation return path 109 for the purpose of simplicity, however, those skilled in the art will appreciate that practical implementations of the bathing unit system 100 may include multiple flow conduits forming multiple primary and/or secondary circulation forward and return paths interconnecting the water inlets 110 and the water outlets 108 of the receptacle 102.

The bathing unit system 100 also includes a set of bathing unit components positioned within the circulation system 106. In the specific embodiment shown in FIG. 1, the set of bathing unit components includes a filter 124, a water pump 112, a heater 116, a sanitizing system 130, and the water quality monitoring system 101. Those skilled in the art will appreciate that practical implementations of the bathing unit system 100 may include additional or fewer bathing unit components that may be positioned in various suitable positions in the circulation system 106.

The sanitizing system 130 may be used for sanitizing the water 104 in the receptacle 102. For example, in the embodiment shown in FIG. 1, the sanitizing system 130 may comprise an automated sanitizer dispensing module 135 configured to dispense a sanitizing agent into the water 104 of the bathing unit system 100. In some embodiments, the automated sanitizer dispensing module 135 may include an electrolytic cell configured to release a free halogen chemical into the water in the water 104 of the bathing unit system 100.

The water quality monitoring system 101 is configured to obtain water quality measurements conveying one or more characteristics of water within the circulation system 106 and transmit the water quality measurements to a cloud-based remote server 180 and/or a controller 122. The remote server 180 and/or the controller 122 may include at least one processor configured to process the water quality measurements. The controller 122 may be located physically near (e.g., in a same room or in a same building as) the bathing unit system 100, while the remote server 180 may be located physically separate (e.g., in a different building from) the bathing unit system 100. In some non-limiting implementations, the water quality monitoring system 101 may be configured to selectively transmit the water quality measurements to the remote server 180 and/or the controller 122 at least in part based on the activation status of the pump 112 when the water quality measurements were obtained and/or transmit the water quality measurements to the remote server 180 and/or the controller 122 with a tag conveying the activation status of the pump 112 when the water quality measurements were obtained. Such embodiments may be desirable as water quality measurements obtained when the pump 112 is deactivated may not be representative of the water quality of the water 104 in the receptacle 102 or of the water 104 in the overall bathing unit system 100. Accounting for the operational status of the pump 112 when the water quality measurements are obtained, for example by omitting certain water quality measurements when the pump 112 was deactivated, may provide for a more accurate measurements of the quality of the water 104 actually circulating through the bathing unit system 100.

In the specific example implementation shown in FIG. 1, the water quality monitoring system 101 includes a water quality monitoring device 140 and a communication module 150. Those skilled in the art will appreciate that practical implementations of the water quality monitoring device 140 may include additional components.

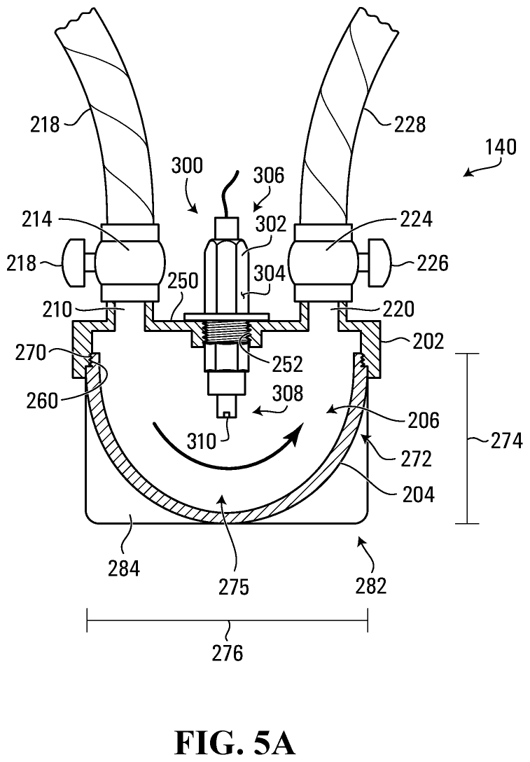

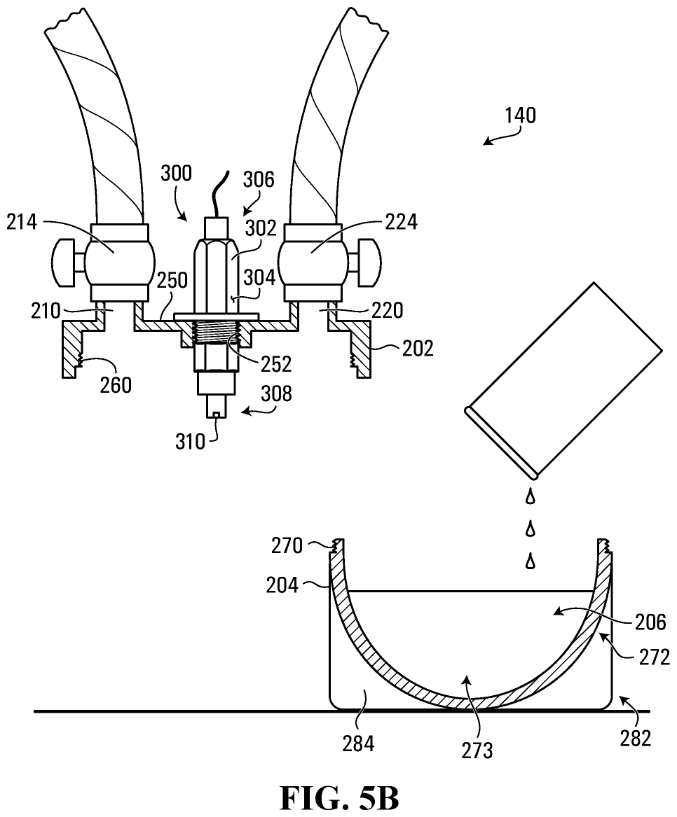

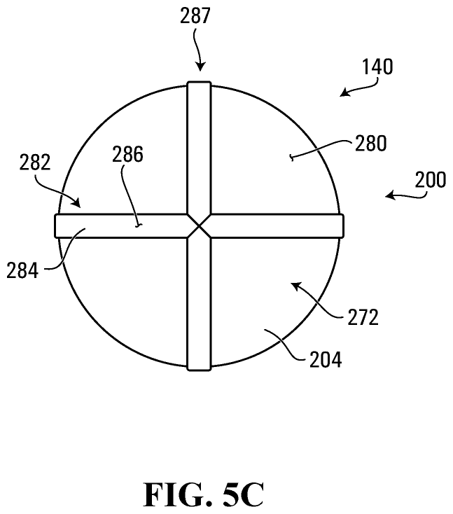

The water quality monitoring device 140 is configured to be positioned inline with the circulation system 106 of the bathing unit system. More specifically, in the example implementation shown in FIG. 1, the water quality monitoring device 140 is positioned inline between the primary circulation forward path 105 and the secondary circulation return path 109. As shown in FIG. 1, the monitoring device 140 includes a probe 300 which includes at least one sensor 310 (shown in FIGS. 5A and 5B) configured to obtain water quality measurements conveying one or more characteristics of water 104 within the circulation system 106. For example, in some embodiments, the sensors 310 may include a pH sensor, an ORP sensor, a turbidity sensor and/or a temperature sensor. Positioning the monitoring device 140 between the primary circulation forward path 105 and the secondary circulation return path 109 may reduce a pressure of the water 104 received within the water quality monitoring device 140 as some of the water 104 from the primary circulation forward path 105 is directed through the primary circulation return path 107.

In the example implementation shown in FIG. 1, the communication module 150 is in communication with the water quality monitoring device 140 for receiving the water quality measurements obtained by the probe 300. The communication module 150 may also be in communication with the controller 122 via communication line(s) 171 or communication network(s) 170 and/or in communication with the remote server 180 via the communication network(s) 170. The communication line(s) 171 may include multiple different types of wired communication lines, such as ethernet, fiber-optical cable, etc. The communication network(s) 170 may can include multiple different types of communication networks, such as a wired or wireless local area network (e.g., a wi-fi network) and/or a public data network such as the internet.

The controller 122 controls the settings/operation of the components in the set of bathing unit components including the settings of the heater 116, the water pump 112, the filter 124, the sanitizing system 130, and/or the water quality monitoring device 140. The bathing unit system 100 also includes a control interface 160 for enabling a user to provide commands for controlling the operational settings of the components in the bathing unit system 100 and optionally for conveying information related to the bathing unit system 100 to the user. The controller 122 receives electrical power from an electric power source (not shown) and controls the distribution of power supplied to the various bathing unit components on the basis of control signals originating from various sensors, program instructions and/or user commands in order to cause desired operational settings to be implemented. Different manners in which the controller 122 may be configured and used to control the bathing unit components for the regulation of the operation of the bathing unit system 100 are generally known in the art and as such will not be described in further detail here. The communication module 150 may receive operational information conveying status information corresponding to operation of the components in the set of bathing unit components, including at least an activation status for the pump 112.

The communication module 150 and the controller 122 are connected to the network(s) 170, enabling the communication module 150 and the controller 122 to communicate with one or more remote devices and/or to access one or more remote services through the network(s) 170. For example, in one non-limiting example of implementation, the communication network(s) 170 includes a (home) Wi-Fi network established by a router 190. More specifically, in this example of implementation, the communication module 150 may communicate with a plurality of remote user devices 182, including for example a smart phone and a laptop, and the remote server 180 through the Wi-Fi network established by the router 190.

In some specific implementations, the communication module 150 may be programmed to: i) selectively transmit the water quality measurements obtained by the water quality monitoring device 140 to the remote server 180 or the controller 122 at least in part by processing the status information of the pump 112 such that at least some water quality measurements obtained by the water quality monitoring device 140 when the pump 112 is inactive are selectively omitted from being transmitted to the remote server 180 or the controller 122; and/or ii) transmit the water quality measurements to the remote server 180 or the controller 122 with a tag conveying the activation status of the pump 112 when the water quality measurements were obtained so that each measurement corresponds to an indication of whether the measurement was taken when the pump 112 was active or inactive. In this way, the remote server 180 or the controller 122 may be able to derive water quality analysis information for the bathing unit system 100 based on water quality measurements that were obtained while the pump 112 was active, while water quality measurements obtained when the pump 112 is inactive are selectively omitted, or de-emphasized relative to the measurements obtained when the pump 112 was active, to derive the water quality analysis information.

For example, in some embodiments, the remote server 180 or the controller 122 may be configured to receive water quality measurements from the communication module 150 that include a tag conveying the activation status of the pump 112 when the water quality measurements were obtained by the water quality monitoring device 140. In such embodiments, the remote server 180 or the controller 122 may be configured to derive the water quality analysis information at least in part by processing the water quality measurements and the tag. Processing the water quality measurements may involve emphasizing water quality measurements taken when the pump 112 was active relative to water quality measurements taken when the pump 112 was inactive, for example by attributing a higher weighting to water quality measurements obtained when the pump 112 was active than when the pump 112 was inactive. Additionally or alternatively, processing the water quality measurements may include selectively discarding the water quality measurements at least in part based on the tag such that water quality measurements obtained when the pump 112 is inactive are selectively omitted from being used to derive the water quality analysis information, which amounts to attributing a “0” weight to measurements obtained when the tag conveys that the measurements were taken when the pump 112 was inactive. In some embodiments, the water quality analysis information includes: i) water quality analysis data for the bathing unit system 100, such as the pH measurements, ORP measurements, turbidity measurements, and/or temperature of the water 104 within the bathing unit system 100; and/or a recommendation to initiate one or more actions in connection with regulating water quality within the bathing unit system 100. In specific implementations, the recommendation may convey an addition of an amount of sanitizer to the bathing unit system 100, an addition of an amount of water the bathing unit system 100, a need for a calibration operation to be performed on the water quality monitoring device 140, a need for a storage operation to be performed on the water quality monitoring device 140, and/or a need for a cleaning operation to be performed on the water quality monitoring device 140.

In some embodiments, the remote server 180 or the controller 122 may be configured to transmit the water quality analysis information for the bathing unit system 100 to the communication module 150 of the water quality monitoring system 101 and/or to one or more of the remote user devices 182 associated with the bathing unit system 100. For example, the water quality analysis information may be configured to cause a notification conveying the water quality analysis data and/or the recommendation(s) to initiate action(s) to be displayed to a user on a display interface 184 of one or more of the remote user devices 182 and/or on a display interface of the control interface 160 of the bathing unit system 100.

In some embodiments, one or more of the remote user devices 182 may run a client application for remotely monitoring and/or controlling the bathing unit system 100 and may be configured to generate and display a graphic user interface on the display interface 184 of the remote user device 182 in order to facilitate that functionality. For example, in some embodiments, the remote user devices 182 may be configured to display a graphic user interface configured to convey operational settings of components in the set of bathing unit components, and notifications indicative of water quality in the bathing unit system 100, such as water condition and chemical levels and/or recommendations based on the identified water condition and identified chemical levels. For example, in some implementations, the remote user devices 182 may run a client application that facilitates the selection of monitoring parameters to configure the water quality monitoring system 101 to monitor and transmit water quality measurements at defined intervals (e.g., daily or hourly) or, in some instances, on a continuous basis.

In some embodiments, the water quality analysis information received from the remote server 180 or generated by the controller 122 itself may be used by the controller 122 to automatically regulate operation of components in the set of bathing unit components to automatically regulate water quality within the bathing unit system 100. For example, in one example implementation, the water quality analysis information from the remote server 180 may be used for automatic regulation of the automated sanitizer dispensing module 135, the heater 116 and/or the pump 112.

### Housing 200

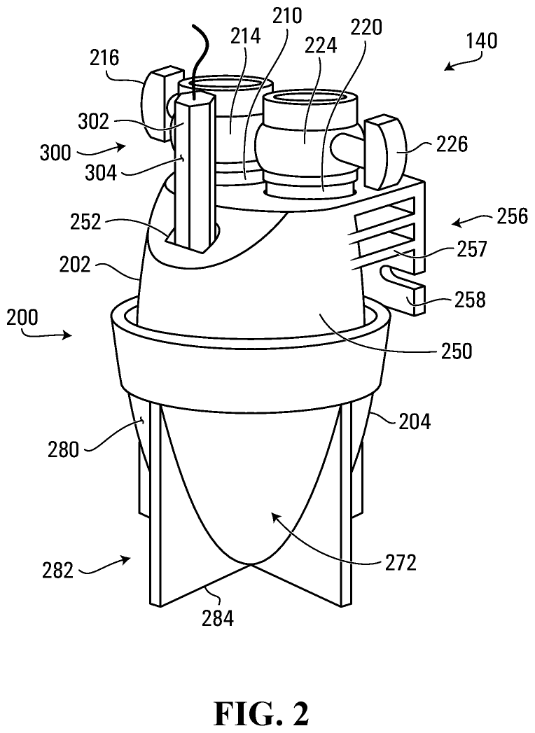

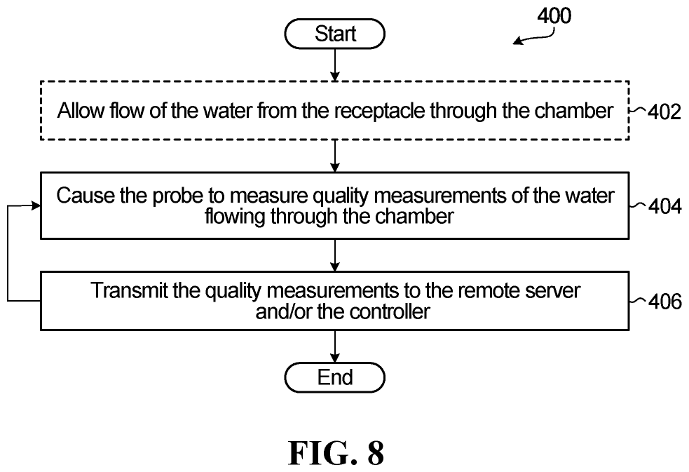

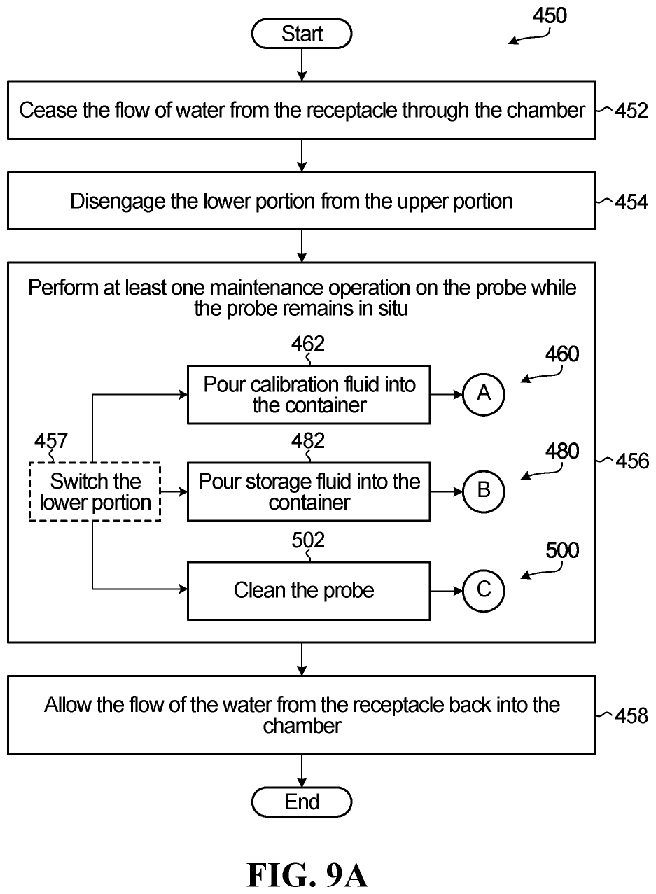

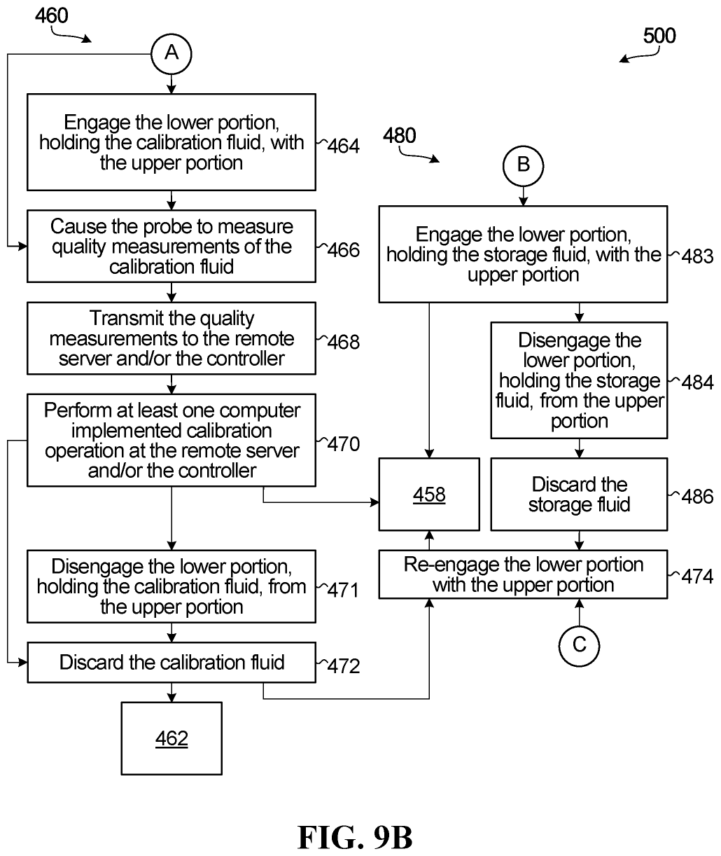

The water quality monitoring device 140 in accordance with one embodiment is shown in FIG. 2. In the embodiment shown, the water quality monitoring device 140 includes a housing 200 and a probe 300 extending into the housing 200. The housing 200 comprises an upper portion 202 and a lower portion 204 releasably engaged with the upper portion 202. When the lower portion 204 is engaged with the upper portion 202, the combination of the upper and lower portions 202 and 204 forms a chamber 206 (shown in FIG. 5A) through which water from the receptacle 102 may flow when the water quality monitoring device 140 is operated in certain operational modes, including the water quality monitoring mode 400 (shown in FIG. 8), as described below. However, when the lower portion 204 is disengaged with the upper portion 202, the lower portion 204 forms a container 208 (shown in FIG. 5B) which may hold a fluid when the water quality monitoring device 140 is operated in different operational modes including one or more maintenance modes 450 (shown in FIGS. 9A and 9B), as described below.

### Inlet Port 210 and Outlet Port 220

The housing 200 is configured to be fluidly coupled to the circulation system 106 and may specifically be fluidly coupled to the receptacle 102 via the circulation system 106 to allow the water 104 from the receptacle 102 to flow through the chamber 206 formed when the lower portion 204 is engaged with the upper portion 202. In the embodiment shown in FIG. 2, the housing 200 includes at least one inlet port 210 (only one inlet port 210 is shown) configured to be in fluid communication with an inlet conduit 218 (e.g., forming a part of the circulation forward path 105) and at least one outlet port 220 (only one outlet port 220 is shown) in fluid communication with an outlet conduit 228 (e.g., forming a part of the secondary circulation return path 109). The inlet conduit 218 is in fluid communication with receptacle 102 and receives the water 104 from the receptacle 102 into the chamber 206; the outlet conduit 228 is also in fluid communication with the receptacle 102 and returns the water 104 within the chamber 206 back into the receptacle 102. Accordingly, the chamber 206 formed when the lower portion 204 is engaged with the upper portion 202 is positioned between the inlet port 210 (the circulation forward path 105) and the outlet port 220 (the secondary circulation return path 109) such that, when the housing 200 is coupled inline in the circulation system 106, at least part of the water 104 from the receptacle 102 enters the chamber 206 through the inlet port 210 and exits the chamber 206 through the outlet port 220.

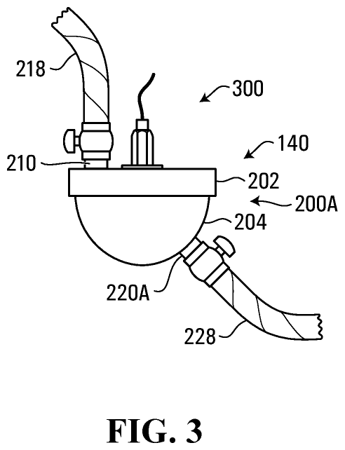

In the embodiment shown in FIG. 2, the inlet port 210 and outlet port 220 are formed in the upper portion 202 of the housing 200. Accordingly, both the inlet port 210 and the outlet port 220 extend upwardly relative to the chamber 206 formed when the lower portion 204 is engaged with the upper portion 202. However, another embodiment of a housing 200A is shown in FIG. 3. In the embodiment shown in FIG. 3, an outlet port 220A may instead be formed in the lower portion 204 of the housing 200A, and may extend downwardly relative to the chamber 206. Such embodiments may facilitate exit of the water 104 within the chamber 206 through the outlet port 220A, due to gravitational forces exerted on the water 104 within the chamber 206 towards the outlet port 220A. Such embodiments may also require dis-engagement of the outlet port 220A from the outlet conduit 228 when the lower portion 204 is disengaged from the upper portion 202. The housing 200A may be used in situations where the inlet conduit 218, the water quality monitoring device 140 and the monitoring outlet conduit 228 form a side flow path (e.g., the secondary circulation return path 109) of the circulation system 106 different from a main flow path (e.g., the primary circulation return path 107) of the circulation system 106 as described above, as water 104 in the side flow path may experience less pressure, which may reduce an amount and a speed of the water 104 which flows through the chamber 206. However, use of the outlet port 220A which extends downwardly relative to the chamber 206 may increase the speed of the water which flows through the chamber 206 due to the gravitational force promoting exit of water through the outlet port 220A. As still further alternatives, the inlet port 210 and the outlet port 220 may both extend laterally from the chamber 206. In such embodiments, the inlet port 210 and the outlet port 220 may both extend in a substantially similar lateral direction or the inlet port 210 and the outlet port 220 port may extend in opposing lateral directions.

Referring back to FIG. 2, the inlet port 210 comprises at least one inlet valve 214 (only one inlet valve 214 is shown) configured to open (for example fully open or proportionally open in some embodiments) and to close the inlet port 210 so as to control the flow of water through the inlet port 210. Similarly, the outlet port 220 also comprises at least one outlet valve 224 (only one outlet valve 224 is shown) configured to open (for example fully open or proportionally open in some embodiments) and to close the outlet port 220, to control flow of water through the outlet port 220. In some embodiments, the inlet and outlet valves 214 and 224 may comprise a combined at least one valve configured to open and close both the inlet and outlet ports 210 and 220. The inlet and outlet valves 214 and 224 and the combined at least one valve may comprise any suitable type of fluid valve known to those skilled in the art. For example, the inlet and outlet valves 214 and 224 and the combined at least one valve may comprise, without being limited to, a gate valve, a ball valve, a check valve, a pinch valve, a butterfly valve, a plug valve, a solenoid valve, etc.

The inlet valve 214 may further comprise an inlet valve lock 216 configured to lock the inlet valve 214 in an open position (for example a fully open position or a partially open position) to allow the chamber 206 to receive the water 104 from the receptacle 102 via the inlet port 210 and the circulation forward path 105, and in a closed position to prevent the chamber 206 from receiving the water 104 from the receptacle 102. The outlet valve 224 also comprises an outlet valve lock 226 configured to lock the outlet valve 224 in an open position (for example a fully open position or a partially open position) to allow the chamber 206 to return the water 104 to the receptacle 102 via the outlet port 220 and the circulation return path 109 and in a closed position to prevent the chamber 206 from returning the water to the receptacle 102. In some embodiments, such as embodiments where the inlet and outlet valves 214 and 224 comprise the combined at least one valve, the inlet and outlet valve locks 216 and 226 may comprise a combined valve lock configured to lock the combined at least one valve, or both the inlet and outlet valves 214 and 224 in the open position (e.g., the fully open or the partially open position) and the closed position. The inlet valve lock 216, the outlet valve lock 226, and the combined at least one valve lock may be each configured to switch between a locked state and an unlocked state in response to electronic actuation events (for example electronic lock or unlock signals) and/or mechanical actuation events. In some specific implementations, the electronic actuation events may be signals transmitted from the controller 122, from another component in the bathing unit system 100, and/or from the remote server 180; the mechanical actuation events may be physical manipulations of locking mechanisms by a user at the inlet and outlet valve locks 216 and 226 or the combined at least one valve lock.

For example, in one embodiment, the inlet and outlet valve locks 216 and 226, the combined at least one valve lock, the inlet and outlet valves 214 and 224 and/or the combined at least one valve may be in communication with the controller 122 and may be responsive to electronic actuation events in the form of signals received from the controller 122 to control flow of water through the chamber 206. For example, the inlet and outlet valve locks 216 and 226 or the combined at least one valve lock may be responsive to a “LOCK” signal from the controller 122 to lock the respective inlet and outlet valves 214 and 224 or the combined at least one valve in the open position or in the close position. The inlet and outlet valve locks 216 and 226 or combined at least one valve lock may be responsive to an “UNLOCK” signal from the controller 122 to unlock the respective inlet and outlet valves 214 and 224 or the combined at least one valve to permit the inlet and outlet valves 214 and 224 or the combined at least one valve to switch from the open position to the close position and vice-versa. In some embodiments, the inlet and outlet valve locks 216 and 226, the combined at least one valve lock, the inlet and outlet valves 214 and 224 and/or the combined at least one valve may also be responsive to a “OPEN” signal from the controller 122, to open the respective inlet and outlet valves 214 and 224 or the combined at least one valve. The inlet and outlet valve locks 216 and 226, the combined at least one valve lock, the inlet and outlet valves 214 and 224 and/or the combined at least one valve may also be responsive to a “x % OPEN” signal from the controller 122, where x may be 25, 50 and 75 for example, to partially open the respective inlet and outlet valves 214 and 224 or the combined at least one valve. Further, the inlet and outlet valve locks 216 and 226, the combined at least one valve lock, the inlet and outlet valves 214 and 224 and/or the combined at least one valve may also be responsive to a “CLOSE” signal from the controller 122, to close the respective inlet and outlet valves 214 and 224 or the combined at least one valve.

In an alternative embodiment, the inlet and outlet valve locks 216 and 226, the combined at least one valve lock, the inlet and outlet valves 214 and 224 and/or the combined at least one valve may include manually operatable locking mechanisms configured for being manually manipulated by a user to lock the respective inlet and outlet valves 214 and 224 or the combined at least one valve in the open position (for example a fully open position or a partially open position) and/or in the closed position. Any suitable manually operatable locking mechanisms may be used here in practical implementations.

In embodiments where the at least one inlet valve 214 comprises a plurality of inlet valves 214 (not shown), the inlet valve lock 216 may comprise a respective inlet valve lock 216 for each of the plurality of inlet valves 214, a single inlet valve lock 216 for the entire plurality of inlet valves 214, or one inlet valve lock 216 for a subset of the plurality of inlet valves 214. In embodiments where the at least one outlet valve 224 comprises a plurality of outlet valves 224 (not shown), the outlet valve lock 226 may be similarly modified.

### Conduit Configurations

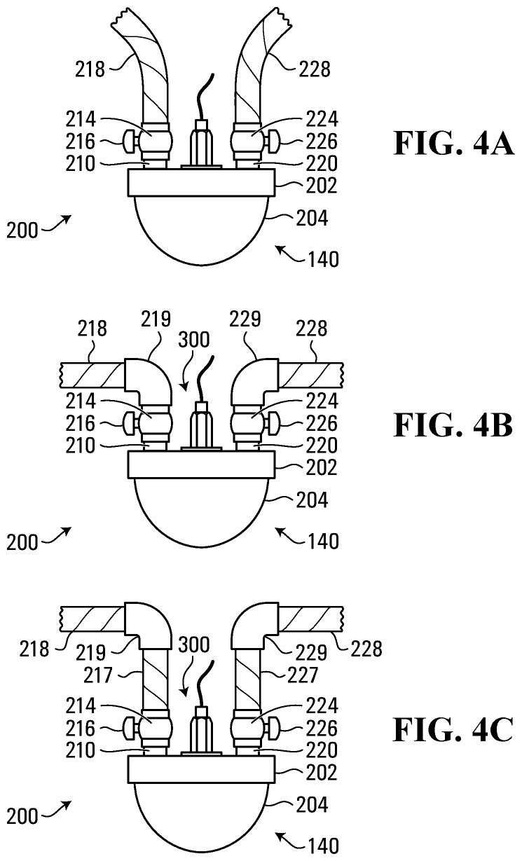

Referring now to FIGS. 4A-4C, the water quality monitoring device 140 may be coupled to the inlet conduit 218 of the circulation forward path 105 and the outlet conduit 228 of the circulation return path 109 in a variety of different configurations. While specific couplings between the water quality monitoring device 140 and the circulation system 106 are described below, those skilled in the art will recognize that the coupling may comprise any coupling which allows the water quality monitoring device 140 to receive the water 104 from the receptacle 102 via the inlet port 210 and to return the water 104 to the receptacle 102 via the outlet port 220.

In the embodiment shown in FIG. 4A, the inlet valve 214 may be coupled directly to the inlet conduit 218 and the outlet valve 224 may be coupled directly to the outlet conduit 228. In another embodiment shown in FIG. 4B, the inlet valve 214 may be directly coupled to an inlet corner fitting 219 which is in turn directly coupled to the inlet conduit 218, while the outlet valve 224 may be directly coupled to an outlet corner fitting 229 which is in turn directly coupled to the outlet conduit 228. In the embodiment shown in FIG. 4B, the inlet and outlet corner fittings 219 and 229 are both 90° corner fittings; however, those skilled in the art will recognize that the inlet and outlet corner fittings 219 and 229 may be configured for a variety of different angles and may range between approximately 45° and approximately 145° for example. In another embodiment shown in FIG. 4C, the inlet valve 214 may be directly coupled to an intermediate inlet conduit 217 which is in tum directly coupled to the inlet corner fitting 219 which is itself in tum directly coupled to the inlet conduit 218. Similarly, the outlet valve 224 may be directly coupled to an intermediate outlet conduit 227 which is in turn directly coupled to the outlet corner fitting 229, which is itself in turn directly coupled to the outlet conduit 228.

### Upper Portion 202 and Lower Portion 204

In the embodiment shown in FIGS. 2, 5A and 5B, the upper portion 202 of the housing 200 comprises a probe retaining component 250 for supporting the probe 300 and for coupling the probe 300 to the upper portion 202 such that the probe 300 extends into the chamber 206 formed when the lower portion 204 is engaged with the upper portion 202 and such that the probe 300 is exposed and remains in situ relative to the upper portion 202 when the lower portion 204 is disengaged from upper portion 202 to form the container 208. As used herein, the phrase “in situ relative to the upper portion 202” means in a generally existing position or a generally original position, that the probe 300 substantially remains in its existing position or original position relative to the upper portion 202 when the lower portion 204 is engaged with, or is disengaged from, the upper portion 202 for example. However, those skilled in the art will recognize that the phrase “in situ relative to the upper portion 202” does not mean no relative movement between the probe 300 and the upper portion 202, as it is possible for a user, the water 104 from the receptacle 102, or even the fluid received in the container 208, to jostle or slightly move the probe 300 relative to the upper portion 202 when the water quality monitoring device 140 is being operated in different operational modes as described below.

Still referring to FIGS. 2, 5A and 5B, the probe retaining component 250 may include at least one retainer feature for retaining the probe 300 relative to the upper portion 202 and to restrict relative movement between the probe 300 and the upper portion 202. While specific embodiments of the probe retaining component 250 and the retainer feature are described below, those skilled in the art will recognize that that the retainer feature may comprise any structure which retains a relative position of, and restricts relative movement between, the probe 300 and the upper portion 202. In the embodiment shown in FIG. 2, the retaining feature comprises an aperture 252 for receiving the probe 300 and through which the probe 300 may be inserted from outside the housing 200 to extend into the chamber 206. At least one of the aperture 252 or the probe 300 may further include a coupling component to reduce movement of the probe 300 relative to the upper portion 202, such as a flange on the probe 300 larger than the aperture 252, a friction engagement feature on a circumferential surface of the aperture 252 which engages an outer surface 304 of a probe body 302 of the probe 300 (or vice versa), mating components associated with both the aperture 252 and the probe 300 (e.g., corresponding magnetic fasteners on the circumferential surface and the outer surface 304, corresponding threads on the circumferential surface and the outer surface 304, corresponding snap-fit fasteners on the circumferential surface and the outer surface 304, etc.) which frictionally engages the outer surface 304 of the probe body 302 with a surface of the upper portion 202. In other embodiments (not shown), the retaining feature and the coupling component may instead comprise features and components located on different parts of the probe 300 and the upper portion 202, such as adhesives, snap-fit fasteners, corresponding threads or other fastener on a lower surface of the probe retaining component 250 and on an upper end 306 of the probe body 302, such that the probe body 302 is internal to the housing 200. In yet other embodiments (not shown), the probe retaining component 250 and the probe body 302 may be integral with each other so as to form a unitary component.

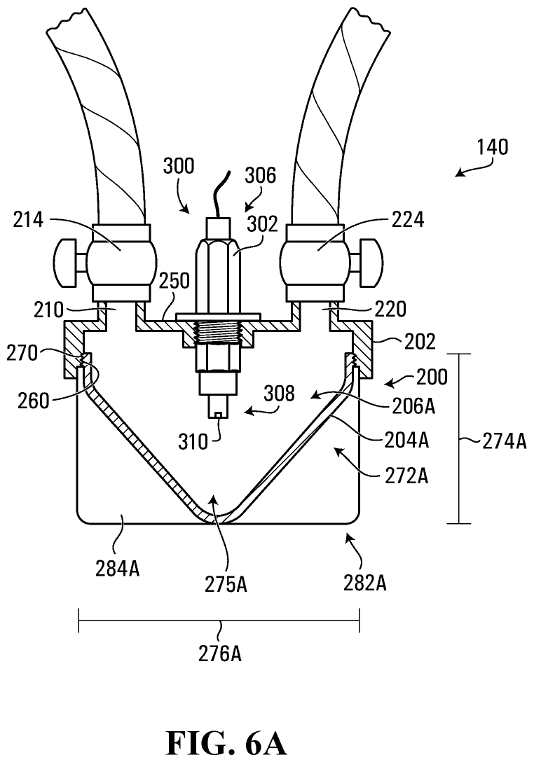

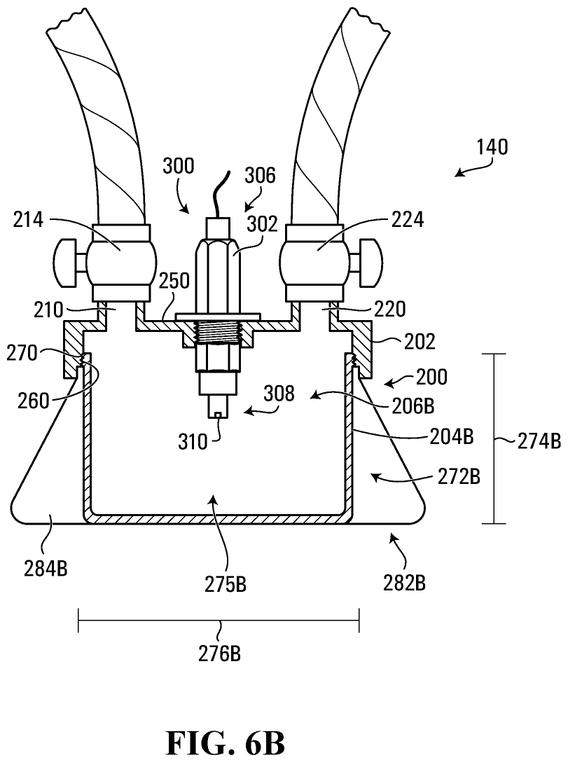

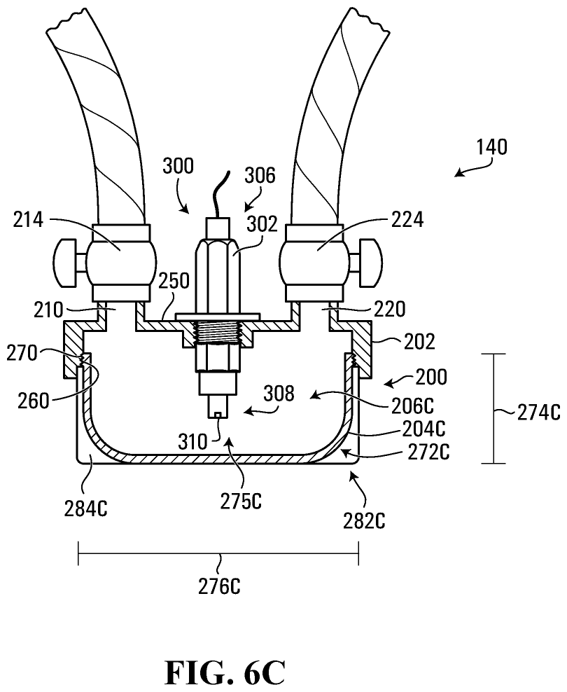

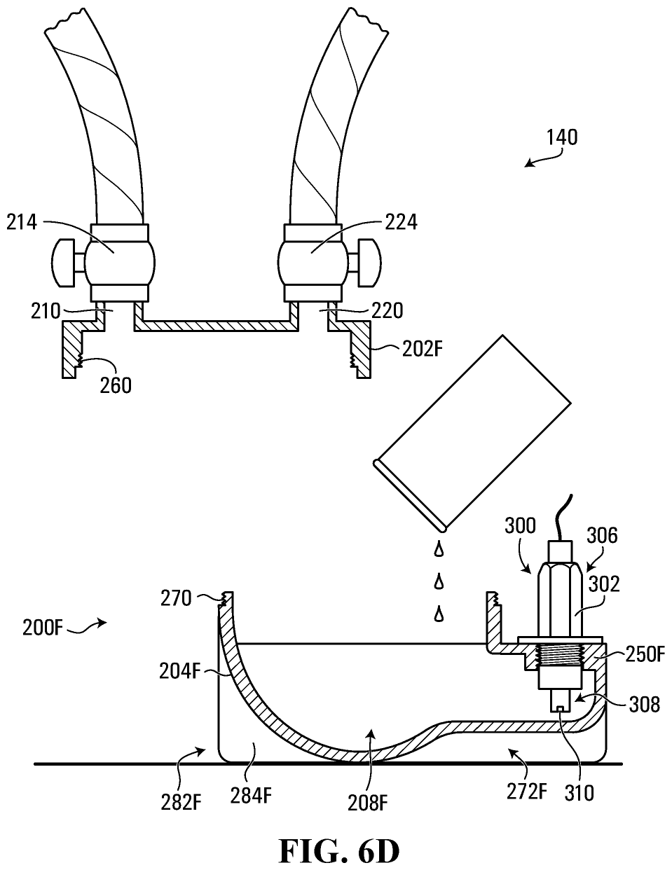

Referring to FIG. 6D, in some embodiments, a lower portion 204F of a housing 200F may comprise a probe retaining component 250F for supporting the probe 300 rather than an upper portion 202F of the housing 200F. The probe retaining component 250F may couple the probe 300 to the lower portion 204F such that the probe 300 extends into a chamber (not shown) formed when the lower portion 204F is engaged with the upper portion 202F and such that the probe 300 also extends into a container 208F and remains in situ relative to the lower portion 204F when the lower portion 204F is disengaged from upper portion 202F to form the container 208F. As used herein, the phrase “in situ relative to the lower portion” means in a generally existing position or a generally original position relative to the lower portion 204F, that the probe 300 substantially remains in its existing position or original position relative to the lower portion 204F when the lower portion 204F is engaged with, or is disengaged from, the upper portion 202 for example. However, those skilled in the art will recognize that the phrase “in situ relative to the lower portion 204F” does not mean no relative movement between the probe 300 and the lower portion 204F, as it is possible for a user, the water 104 from the receptacle 102, or even the fluid received in the container 208F, to jostle or slightly move the probe 300 relative to the lower portion 204F when the water quality monitoring device 140 is being operated in different operational modes as described below. The probe retaining component 250F may also include at least one retaining feature for retaining the probe 300 relative to the lower portion 204F and to restrict movement of the probe 300 relative to the lower portion 204F. The at least one retaining feature of the probe retaining component 250F of the lower portion 204F may be similar to the at least one retaining feature of the probe retaining component 250 of the upper portion 202.

Referring back to FIG. 2, the upper portion 202 further comprises a support component 256 extending from the probe retaining component 250 for fixedly coupling the upper portion 202 to a supporting structure of the bathing unit system 100. While specific embodiments of the support component 256 are described below, those skilled in the art will recognize that that the support component 256 may comprise any structure for fixedly coupling the upper portion 202 to the supporting structure in the bathing unit system 100. In the embodiment shown in FIG. 2, the support component 256 includes one or more horizontal ribs 257 extending from the probe retaining component 250 and a vertical flange 258 extending from the horizontal ribs 257 including a slot or an aperture for receiving a faster to fixedly coupled to the vertical flange 258 to a wall of the bathing unit system 100. In other embodiments, the support component 256 may comprise other structures operable for fixedly coupling the upper portion 202 to the bathing unit system 100, such as a set of screws, a magnetic, adhesive or snap-fit component configured to couple with a corresponding magnetic, adhesive or snap-fit component on the wall of the bathing unit system 100. Generally, when the upper portion 202 is fixedly coupled to the bathing unit system 100 via the support component 256, the probe 300 retained by the probe retaining component 250 is also configured to remain in situ relative to the bathing unit system 100 (in addition to remain in situ relative to the upper portion 202) when the lower portion 204 is disengaged with upper portion 202. Again, those skilled in the art will recognize that the phrase “in situ relative to the bathing unit system 100” means that the probe 300 substantially remains in its existing position or original position relative to the bathing unit system 100 when the lower portion 204 engaged to, or disengaged from, the upper portion 202; however the phrase does not that there is no relative movement between the probe 300 and the bathing unit system 100, as it is possible for a user, the water 104, or even the fluid to jostle or slightly move the probe 300 relative to bathing unit system 100 when the water quality monitoring device 140 is being operated in different operational modes as described below.

The supporting structure of the bathing unit system 100 to which the upper portion 202 is coupled may comprise a side wall of the receptacle 102 or an internal wall of a frame structure of the bathing unit system 100. In embodiments where the bathing unit system 100 is a spa system, this supporting structure of the bathing unit system 100 may be located underneath a skirt (not shown) of the bathing unit system 100 to hide the water quality monitoring device 140. The skirt of the bathing unit system 100 may include a suitably positioned access panel or other door structure (not shown) to allow the user to access the water quality monitoring device 140 when the water monitoring device is being operated in different operational modes as described below.

Referring now to FIGS. 5A and 5B, the upper portion 202 further comprises an upper engagement component 260 extending from the probe retaining component 250 and configured to releasably engage with a corresponding lower engagement component 270 of the lower portion 204 for releasably engaging the upper portion 202 and the lower portion 204. While specific embodiments of the upper and lower engagement components 260 and 270 are described below, those skilled in the art will recognize that that the upper and lower engagement components 260 and 270 may comprise any suitable structure for releasably engaging lower portion 204 with the upper portion 202. Referring to FIGS. 5A and 5B, in the embodiment shown, the upper engagement component 260 comprises inner threads on the upper portion 202 and the lower engagement component 270 comprises corresponding outer threads on the lower portion 204 which may be releasably engaged with each other using rotational force applied in opposing directions. However, in other embodiments, the upper and lower engagement components 260 and 270 may comprise alternative rotational coupling features, including corresponding bayonet features on the upper and lower portions 202 and 204. The upper and lower engagement components 260 and 270 may also comprise corresponding frictional coupling features (e.g., corresponding snap-fit features on the upper and lower portions 202 and 204) and corresponding magnetic coupling features (e.g., a magnet having a first polarity on the upper portion 202 and a magnet having a second polarity opposite the first polarity on the lower portion 204). In yet other embodiments, the upper and lower engagement components 260 and 270 may comprise a single unified engagement component, such as an external clamp for frictionally coupling the upper and lower portions 202 and 204.

The lower portion 204 comprises a body component 272 extending from the lower engagement component 270. The body component 272 is shaped and dimensioned to define a shape and a container volume 273 (shown in FIG. 5B) of the container 208 when the lower portion 204 is disengaged from the upper portion 202 (e.g., the upper and lower engagement components 260 and 270 are disengaged from each other). The shape and dimension of the body component 272 also cooperates with the upper portion 202 to also to define a shape and a chamber volume 275 (shown in FIG. 5A) of the chamber 206 when the lower portion 204 is engaged with the upper portion 202 (e.g., the upper and lower engagement components 260 and 270 are engaged with each other). In some embodiments, the body component 272 may also define an area for retaining the probe 300 (shown in FIG. 6D). While specific embodiments of the body component 272 are described below, those skilled in the art will recognize that that the body component 272 may have any suitable shape and any suitable dimension which enables the body component 272 to define the container 208 operable to receive a fluid as described below and to define the chamber 206 operable to receive the water 104 from the receptacle 102 as described above and below.

For example, in the embodiment shown FIGS. 5A and 5B, the body component 272 has a dome shape, a body height 274 and a base body width 276 to define the container 208 with a U-shaped cross-section and the container volume 273 for holding fluid when the lower portion 204 is disengaged from the upper portion 202 and when the water quality monitoring device 140 is being operated in certain operational modes as described below. Similarly, referring to FIG. 5A, the dome shape, the body height 274 and the base body width 276 also define the chamber 206 with a U-shaped cross-section and the chamber volume 275 through which the water 104 from the receptacle 102 may flow when the lower portion 204 is engaged with the upper portion 202 and the water quality monitoring device 140 is being operated in other operational modes as described below.

In other embodiments, the body component 272 may be shaped differently such that the container 208 and the chamber 206 have different shapes than described above. For example, another embodiment of a lower portion 204A comprising a body component 272A is shown in FIG. 6A. The body component 272A has a conical shape, a body height 274A and a base body width 276A to define a) a container (not shown) having a V-shaped cross-section and a container volume when the lower portion 204A is disengaged from the upper portion 202 and b) a chamber 206A having a V-shaped cross-section and a chamber volume 275A when the lower portion 204A is engaged with the upper portion 202. The containers and chambers 208, 206 and 206A having the U-shaped or the V-shaped cross-sections can reduce the corresponding container or chamber volumes 273, 275, 275A while maintaining the body heights 274 and 274A which allow extension of the probe 300 into the chambers 206 and 206A. However, in other embodiments, the body component 272 may be shaped to have a consistent body width 276 throughout the body height 274. For example, another embodiment of a lower portion 204B is shown in FIG. 6B. The body component 272B has a cylindrical shape, a body height 274B and a base body width 276A to define a) a container (not shown) having a rectangular-shaped cross-section and a container volume when the lower portion 204B is disengaged from the upper portion 202 and b) a chamber 206B also having a rectangular-shaped cross-section and a chamber volume 275B when the lower portion 204B is engaged with the upper portion 202.

In other embodiments, the body component 272 may be dimensioned differently such that the container 208 and the chamber 206 have different dimensions and/or volumes than described above. For example, another embodiment of a lower portion 204C comprising a body component 272C is shown in FIG. 6C. The body component 272C has a dome shape, a body height 274C and a base body width 276C to define a) a container (not shown) having a U-shaped cross-section and a container volume when the lower portion 204C is disengaged from the upper portion 202 and b) a chamber 206C also having a U-shaped cross-section and a chamber volume 275C when the lower portion 204C is engaged with the upper portion 202. The dome shape and the base body width 276C of the body component 272C may be similar to the dome-shape and the base body width 276 of the body component 272 (shown in FIG. 5A); however the body height 274C may be smaller than the body height 274 (again, shown in FIG. 5A), and may be 90%, 80%, 75% or 50% of the body height 274, which in turn reduces the container and chamber volumes 275C relative to the container and chamber volumes 273 and 275. The reduced body height 274C and the reduced container volume can allow the sensor end 308 of the probe 300 to be fully submerged when a smaller volume of fluid is received in the container when the lower portion 204C is re-engaged with the upper portion 202. This can reduce the amount of fluid required in order to perform certain maintenance operations when the water quality monitoring device 140 is operated in one or more maintenance modes 450 (shown in FIGS. 9A and 9B).

In some embodiments, the water quality monitoring device 140 may include a plurality of lower portions 204 which may be interchangeable with one another, and which may provide for different configurations of the housing 200 when the water quality monitoring device 140 is being operated in different operational modes. In such embodiments, the upper portion 202 of the housing 200 (and the probe 300 retained by the upper portion 202 in some embodiments) may generally remain in an existing position or an original position while the lower portion 204 is disengaged therefrom and exchanged for an alternative lower portion 204. As a more specific example, when the water quality monitoring device 140 is being operated in the water quality monitoring mode 400 (shown in FIG. 8), the lower portion 204 shown in FIGS. 5A and 5B may be engaged with the upper portion 202; however, when the water quality monitoring device 140 is being operated in one of the maintenance modes 450 (shown in FIGS. 9A and 9B), the lower portion 204A shown in FIG. 6A or the lower portion 204C shown in FIG. 6C may be used to reduce the container and chamber volumes 273 and 275, which may in turn reduce the amount of storage fluid, calibration fluid or cleaning fluid required for the maintenance modes 450.

Referring back to FIG. 2, the lower portion 204 may further comprise an outer surface 280 and a grip component 282 outwardly extending from the outer surface 280. The grip component 282 may be shaped and dimensioned to facilitate manipulation of the lower portion 204 using a hand of a user in order to disengage the lower portion 204 from the upper portion 202 (e.g., disengage the upper and lower engagement components 260 and 270 from each other) and also to re-engage the lower portion 204 to the upper portion 202 (e.g., engage the upper and lower engagement components 260 and 270 to each other). In some embodiments, the grip component 282 may further be shaped and dimensioned to allow the container 208 defined by the lower portion 204 to remain upright when placed on a level surface. While specific embodiments of the grip component 282 are described below, those skilled in the art will recognize that that the grip component 282 may comprise any structure operable to facilitate manual manipulation of the lower portion 204 to disengage the lower portion 204 from the upper portion 202 and/or which is operable to allow the lower portion 204 to remain upright when placed on a level surface. Some embodiments of the lower portion 204 may not include the grip component 282.

In the embodiment shown in FIGS. 2 and 5A-5C, the grip component 282 comprises a plurality of vane grips 284. Each vane grip of the plurality of vane grips 284 comprises a vane body configured to be grasped by fingers of a user to facilitate manipulation of the lower portion 204. Each vane body comprises a flat bottom surface 286 (best shown in FIG. 5C) which supports the container 208 and allows the container 208 to remain upright when the lower portion 204 is placed on a level surface (shown in FIG. 5B). Additionally, in the embodiment shown, the plurality of vane grips 284 comprises four vane grips 284, including two sets of opposing vane grips 284 which form a cross-shaped support structure 287 (best shown in FIG. 5C). The vane body and the bottom surface 286 of each vane grip 284 may be shaped and dimensioned to support the shape and the dimension of the lower portion 204. In this regard, in the embodiment shown in FIGS. 2 and 5A-5C, each vane grip of the plurality of vane grips 284 may be shaped and dimensioned such that an overall width of the lower portion 204 is approximately the same as the base body width 276 (shown in FIG. 5A) to distribute the weight of the body component 272 so that the lower portion 204 may remain upright when placed on the level surface.

In other embodiments, the vane body and the bottom surface 286 of each vane grip 284 may be shaped and dimensioned differently depending on the shape and the dimension of the lower portion 204. For example, referring to FIG. 6A, in embodiments of the lower portion 204A where the body component 272A defines the V-shaped container and chamber 206A, a grip component 282A of the lower portion 204A may define a plurality of vane grips 284A which have a larger vane body when compared to the plurality of vane grips 284 of the lower portion 204 shown in FIGS. 5A and 5B. As another example, referring now to FIG. 6B, in embodiments of the lower portion 204B where the body component 272B defines the rectangular-shaped container and chamber 206B, a grip component 282B of the lower portion 204B may define a plurality of vane grips 284B which extend outwardly such that the overall width of the lower portion 204B is a larger than the base body width 276B. Such embodiments may be used to more securely distribute the weight of the body component 272B and may reduce the likelihood that the lower portion 204B will be tipped over during use. Additionally, in some embodiments (not shown), such as when the body component 272B defines the rectangular-shaped container and chamber 206B as shown in FIG. 6B, the lower portion 204B may not include the grip component 282B as a bottom of the body component 272B may be flat and may itself allow the lower portion 204B to remain upright when positioned on the level surface. As another example, referring to FIG. 6C, in embodiments of the lower portion 204C where the body component 272C defines the shallower U-shaped container and chamber 206C, a grip component 282C of the lower portion 204C may define a plurality of vane grips 284C which have a smaller vane body when compared to the plurality of vane grips 284 of the lower portion 204 shown in FIGS. 5A and 5B, due to the reduced curvature of the body component 272C for example. In some embodiments (not shown), such as when the lower portion 204C defines the shallower U-shaped container and chamber 206C as shown in FIG. 6C, the lower portion 204C may also not include the grip component 282C, as a bottom of the body component 272C may itself be flat enough and wide enough to allow the lower portion 204C to remain upright when positioned on the level surface. As a still further example, referring to FIG. 6D, in embodiments of the lower portion 204F where the body component 272F defines an irregularly shaped container 208F and chamber, a grip component 282F of the lower portion 204F may similarly define a plurality of vane grips 284F having an irregular shape. For example, the vane grips 284F extending from a regular curved portion of the body component 272F (e.g., left side of the body component 272F shown in FIG. 6D) may be similar to the vane grips 284 of the lower portion 204 shown in FIGS. 5A and 5B; however, the vane grips 284F extending from an extended portion of the body component 272F (e.g., right side of the body component 272F shown in FIG. 6D) may be similarly extended in order to support the extended portion of the body component 272F on the level surface and to allow the lower portion 204F to remain upright when positioned on the level surface.

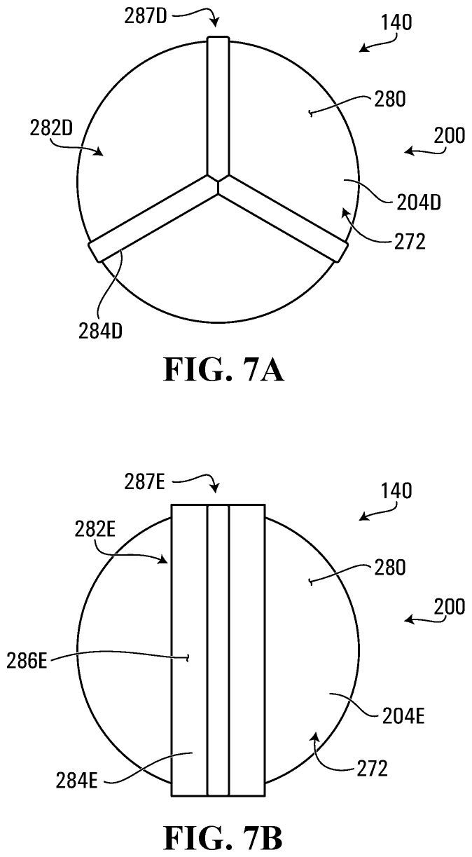

Additionally, in yet other embodiments, the plurality of vane grips 284 may comprise a different number of vane grips. For example, referring to FIG. 7A, another embodiment of a lower portion 204D may include a grip component 282D which comprises three vane grips 284D forming a triangular support structure 287D. As another example, referring to FIG. 7B, a further embodiment of a lower portion 204E may include a grip component 282E which comprises two vane grips 284E forming a linear support structure 287E. Each of the two vane grips 284E may have an expanded bottom surface 286E which to better distribute the weight of the body component 272 so that the lower portion 204E remains upright when placed on the level surface.

The housing 200 may be formed of any type of suitable material for holding the fluid received in the container 208 when the water quality monitoring device 140 is being operated in the maintenance modes 450 as described below and for receiving the water 104 from the receptacle 102 when the water quality monitoring device 140 is being operated in the water quality monitoring mode 400 as described below. For example, the housing 200 may be formed of a metal-based material, a plastic-based material or a glass-based material. The upper portion 202 and the lower portion 204 may be made of a same type material or may be made of different types of material. For example, the upper portion 202 may be formed of a more durable metal-based material as it may be more cumbersome to replace the upper portion 202, while the lower portion 204 may be made of a more cost-effective plastic-based material as the lower portion 204 may be replaced more easily. Additionally, the lower portion 204 may be made of a transparent or translucent material such that the container 208 and/or the chamber 206 is also correspondingly transparent or translucent to allow a user to visualize the probe 300 and the water 104 from the receptacle 102 and/or the fluid in the chamber 206 without disengaging the lower portion 204 from the upper portion 202.

### Probe 300

Referring back to FIGS. 2, 5A and 5B, as briefly described above, the probe 300 includes the probe body 302 defining the upper end 306 and the sensor end 308 and having the outer surface 304. The sensor end 308 includes the at least one sensor 310 configured to obtain quality measurements conveying one or more characteristics of the water 104 received from the receptacle 102 in the chamber 206 when the water quality monitoring device 140 being operated in the water quality monitoring mode 400 (shown in FIG. 8) as described below, or quality measurements convey one or more characteristics of the fluid held in the container 208 when the water quality monitoring device 140 is being operated in one of the maintenance modes 450 (shown in FIGS. 9A and 9B) as described below. As described above, the sensors 310 may include at least one of the pH sensor configured to sense a pH level of the water 104 or the fluid, the ORP sensor configured to sense an ORP level of the water 104 or the fluid, a turbidity sensor configured to sense a turbidity level of the water 104 or the fluid or a temperature sensor configured to sense a temperature of the water 104 or the fluid. In some embodiments, the sensors 310 may include only the pH sensor and the ORP sensor.

In the embodiment shown, the probe 300 may be in communication with the controller 122 and may be responsive to signals received from the controller 122 or another component in the set of bathing unit components to begin measurement of the quality measurements or to cease measurement of the quality measurements. For example, the probe 300 may be responsive to an “ON” signal from the controller 122 to begin obtaining the quality measurements of the water 104 or the fluid and an “OFF” signal from the controller 122 to cease obtaining the quality measurements of the water 104 or the fluid. In some embodiments, the “ON” signal may direct the probe 300 to continuously measure the quality measurements (e.g., the probe 300 may measure the quality measurements in real-time until a subsequent “OFF” signal is received). In other embodiments, the “ON” signal may direct the probe 300 to periodically measure the quality measurements (e.g., the probe 300 may measure the quality measurements every second, every five seconds, every 10 seconds, every 30 seconds, every minute, etc., until a subsequent “OFF” signal is received). In yet other embodiments, the probe 300 may also be responsive to a “CALIBRATE” signal, for example a “CALIBRATE” signal received from the controller 122 or from another component in the set of bathing unit components, to direct the probe 300 to perform a calibration operation to gauge its quality measurements with known quality measurements of a calibration fluid. The calibration operation may involve operating at least one particular sensor of sensors 310 for a calibration period. This calibration period may be five seconds, 10 seconds, 30 seconds, 60 seconds, etc. In some embodiments, each sensor of the sensors 310 may be calibrated individually with at least one corresponding calibration fluid held in the container 208 (e.g., to calibrate the pH sensor, the calibration fluid may comprise a 4.01 pH calibration fluid and a 7.01 pH calibration fluid; to calibrate the ORP sensor, the calibration fluid may instead comprise a 255 mV ORP calibration fluid). In other embodiments, more than one sensor of the sensors 310 may be calibrated together using a same calibration fluid (e.g., to calibrate both the pH sensor and the ORP sensor, the calibration fluid may comprise a 255 mV ORP and a 4.01 pH calibration fluid).

The probe 300 may communicate the quality measurements of the water 104 and/or the fluid obtained by the sensors 310 to the communication module 150 for transmittal to the remote server 180 and/or to the controller 122 via the communication network(s) 170 and/or the communication line(s) 171 as described above. In some embodiments, the quality measurements may be transmitted continuously by the communication module 150 (e.g., the communication module 150 may transmit the quality measurements as a continuous stream in real-time). In other embodiments, the communication module 150 may periodically transmit the quality measurements (e.g., the communication module 150 may transmit the quality measurements every second, every five seconds, every 10 seconds, every 30 seconds, every minute, etc.). The water quality measurements transmitted by the communication module 150 to the remote server 180 and/or the controller 120 may be tagged with an activation status of the pump 112 and/or may be selectively transmitted based on the activation status of the pump 112 as described in co-pending United States application filed Oct. 27, 2023 and titled METHOD, APPARATUS AND SYSTEM FOR MONITORING WATER QUALITY IN A BATHING UNIT SYSTEM, the contents of which is incorporated by reference in its entirety.

### Water Quality Monitoring Mode 400

As mentioned above and referring to FIG. 8, the water quality monitoring device 140 may be operated in the water quality monitoring mode 400 in some embodiments. For example, in certain embodiments, the controller 122 or other components of the bathing unit system 100 may periodically and automatically initiate operation of the water quality monitoring device 140 in the water quality monitoring mode 400 and may continue operation in the water quality monitoring mode 400 for a monitoring period. Alternatively or additionally, a user may initiate operation of the water quality monitoring device 140 in the water quality monitoring mode 400 through a user operable input. The user operable input may be provided in the form of an actuator (not shown) on the water quality monitoring device 140, of an actuator displayed or otherwise present on the control interface 160 (shown in FIG. 1) or of an actuator displayed on one or more of the remote user devices 182 (also shown in FIG. 1). While specific embodiments of operating the water quality monitoring device 140 in the water quality monitoring mode 400 are described below in association with the flowchart illustrated in FIG. 8, other methods of operating the water quality monitoring device 140 in the water quality monitoring mode 400 may alternatively be used. For example, the order of execution of the blocks in FIG. 8 may be altered, and/or some of the blocks described may be altered, eliminated, or combined.

While the water quality monitoring device 140 is being operated in the water quality monitoring mode 400, the lower portion 204 may be engaged with the upper portion 202 to form the chamber 206 (shown in FIG. 5A). Operating the water quality monitoring device 140 in the water quality monitoring mode 400 may begin at optional block 402, which may involve allowing flow of the water 104 from the receptacle 102 through the chamber 206. For example, block 402 may involve causing the controller 122 to transmit the “OPEN” or the “x % OPEN” signal to the inlet and outlet valves 214 and 224 or the combined at least one valve (and optionally the inlet and outlet valve locks 216 and 226 or the combined at least one valve lock) to direct the inlet and outlet valves 214 and 224 or the combined at least one valve to open (or partially open) the respective inlet and outlet ports 210 and 220 (and optionally to direct the inlet and outlet valve locks 216 and 226 or the combined at least one valve lock to lock the respective inlet and outlet ports 210 and 220 in the open position (or the partially open position)). This allows the water 104 from the receptacle 102 to flow into the chamber 206 via the inlet port 210, through the chamber 206, and out of the chamber 206 via the outlet port 220 to return to the receptacle 102.

Operating the water quality monitoring device 140 in the water quality monitoring mode 400 may then proceed to block 404, which may involve causing the probe 300 to measure quality measurements of the water 104 flowing through the chamber 206. For example, block 404 may involve causing the controller 122 to transmit the “ON” signal to the probe 300 to cause the sensors 310 to begin measurement of the quality measurements of the water 104 flowing through the chamber 206.

Operating the water quality monitoring device 140 in the water quality monitoring mode 400 may then proceed to block 406, which may involve causing the probe 300 to transmit the quality measurements of the water 104 flowing through the chamber 206 to the communication module 150. The communication module 150 may then transmit the quality measurements to the remote server 180 and/or the controller 122 via the communication network(s) 170 and/or the communication line(s) 171 as described above. As also described above, the quality measurements transmitted by either the probe 300 or by the communication module 150 may be tagged with an activation status of the pump 112 or may be selectively transmitted to the remote server 180 and/or the controller 122 based on the activation status of the pump 112. The transmitted quality measurements of the water 104 flowing through the chamber 206 may then be processed by the remote server 180 and/or the controller 122 to generate water quality analysis information to determine whether the water 104 within the circulation system 106 is of acceptable chemical composition or sanitary conditions or if an intervention (user-implemented and/or automated via automatic control of different components of the bathing unit system 100 (e.g., the automated sanitizer dispensing module 135, the heater 116 and/or the pump 112)) is required.

### One or More Maintenance Modes 450

Referring now to FIGS. 9A and 9B, the water quality monitoring device 140 may also be operated in one or more maintenance modes 450 in which at least one maintenance operation may be performed on the probe 300 while the probe 300 remains in situ relative to the upper portion 202 as shown in FIG. 5B (or in situ relative to the lower portion 204F as shown in FIG. 6D). The one or more maintenance modes 450 may comprise operating the water quality monitoring device 140 in one or more of a calibration mode 460, a storage mode 480 and a cleaning mode 500. Generally, a user may initiate operation of the water quality monitoring device 140 in the maintenance modes 450 through a user operable input. The user operable input may be provided in the form of an actuator (not shown) on the water quality monitoring device 140, of an actuator displayed or otherwise present on the control interface 160 (shown in FIG. 1) or of an actuator displayed on one or more of the remote user devices 182 (also shown in FIG. 1). While specific embodiments of operating the water quality monitoring device 140 in one or more of the maintenance modes 450 are described below in association with the flowcharts illustrated in FIGS. 9A and 9B, other methods of operating the water quality monitoring device 140 in one or more of the maintenance modes 450 may alternatively be used. For example, the order of execution of the blocks in FIGS. 9A and 9B may be altered, and/or some of the blocks described may be altered, eliminated, or combined.

Operating the water quality monitoring device 140 in one or more maintenance modes 450 may begin at block 452, which may involve ceasing flow of the water 104 from the receptacle 102 through the chamber 206. For example, block 452 may involve causing the controller 122 to transmit the “CLOSE” signal to the inlet and outlet valves 214 and 224 or the combined at least one valve (and optionally the inlet and outlet valve locks 216 and 226 or the combined at least one valve lock) to direct the inlet and outlet valves 214 and 224 or the combined at least one valve to close the inlet and outlet ports 210 and 220 (and optionally to direct the inlet and outlet valve locks 216 and 226 or the combined at least one valve lock to lock the inlet and outlet ports 210 and 220 in the closed position). This may prevent any water 104 from the receptacle 102 from flowing into the chamber 206 during one or more of the maintenance modes 450. This may also prevent any of the fluid which may be received in the container 208 during one or more of the maintenance modes 450 as described below to flow out of the chamber 206 and into the receptacle 102 and/or the circulation system 106 of the bathing unit system 100. For example, calibration fluids used during the calibration mode 460, storage fluids used during the storage mode 480 and cleaning fluids used during the cleaning mode 500 may include chemical components that could be harmful to components of the bathing unit system 100 and/or to users of the bathing unit system 100 or which would otherwise not be desirable to introduce into the bathing unit system 100. It may thus be desirable to reduce the likelihood of discharge of such fluids from the chamber 206 into the receptacle 102 after one or more of the maintenance modes 450.

Referring now to FIGS. 5B and 9A, operating the water quality monitoring device 140 in one or more maintenance modes 450 may then proceed to block 454, which may involve disengaging the lower portion 204 from the upper portion 202 to expose the probe 300 while the probe 300 remains in situ relative to the upper portion 202. The exposed probe 300 may be accessed by a user to perform at least one maintenance operation thereon. In other embodiments, and referring to FIGS. 6D and 9A, block 454 may involve disengaging the lower portion 204F from the upper portion 202F such that the probe 300 extends into the container 208F and remains in situ relative to the lower portion 204F. The combination of the disengaged lower portion 204F and the probe 300 may allow a user to perform at least one maintenance operation on the probe 300. For example, block 454 may involve a user manually manipulating at least one of the body component 272 or the grip component 282 of the lower portion 204 to disengage the upper and lower engagement component 260 and 270 from each other. Once disengaged, the lower portion 204 may form the container 208. In some embodiments, block 454 may also involve a user discarding any of the water 104 from the receptacle 102 which remains within the container 208 after the lower portion 204 is disengaged from the upper portion 202. Additionally or alternatively, in some embodiments, block 454 may also involve resting the container 208 formed by the lower portion 204 on the level surface by using the grip component 282 to support the container 208 in an upright position so that the container 208 may receive and hold a fluid used for one or more of the maintenance modes 450 while resting on the level surface.

Referring back to FIGS. 5B and 9A, operating the water quality monitoring device 140 in one or more maintenance modes 450 may then proceed to block 456, which may involve performing at least one maintenance operation on the probe 300 while the probe 300 is exposed and remains in situ relative to the upper portion 202. In other embodiments, and referring to FIGS. 6D and 9A, block 456 may instead involve performing at least one maintenance operation on the probe 300 while the probe 300 extends into the container 208F and remains in situ relative to the lower portion 204F. In one specific embodiment, block 456 may involve operating the water quality monitoring device 140 in one or more of three different maintenance modes including the calibration mode 460, the storage mode 480 and the cleaning mode 500. A particular instance of block 456 may involve only a single maintenance mode (e.g., a user may operate the water quality monitoring device 140 in only the calibration mode 460 before proceeding to block 458 as described below) or may involve more than one maintenance mode (e.g., a user may operate the water quality monitoring device 140 in the cleaning mode 500 first before operating the water quality monitoring device 140 in the calibration mode 460 and/or in the storage mode 480 before proceeding to block 458 as described below).

### Calibration Mode 460

Referring to FIGS. 9A and 9B, in some embodiments, operating the water quality monitoring device 140 in the calibration mode 460 may begin at optional block 457, which may involve switching the lower portion 204 disengaged from the upper portion 202 for another lower portion 204. For example, in some embodiments, block 457 may involve switching the lower portion 204 used when the water quality monitoring device 140 is operated in the water quality monitoring mode 400 (shown in FIGS. 5A and 5B) for the lower portion 204A having the smaller container volume (shown in FIG. 6A) or the lower portion 204C having the smaller container volume (shown in FIG. 6C). Switching the lower portion 204 for another lower portion 204A, 204C having the smaller container volume can reduce the volume of calibration fluid required to submerge and calibrate the probe 300 as described below. Generally, the smaller container volumes may require a smaller volume of calibration fluid in order to achieve a fluid depth sufficient to submerge the probe 300, and use of a smaller volume of calibration fluid is desirable as the calibration fluid may be costly.

Operating the water quality monitoring device 140 in the calibration mode 460 may then proceed to block 462, which may involve pouring at least one calibration fluid into the container 208 formed by the lower portion 204 (which may be a different container formed by a different lower portion 204A, 204C) when it is disengaged from the upper portion 202. The at least one calibration fluid may a fluid having known quality measurements to calibrate the quality measurements measured by the sensors 310 of the probe 300. As briefly described above, in some embodiments, the at least one calibration fluid may comprise one or more corresponding calibration fluids for calibrating a single sensor of the sensors 310 individually. For example, to calibrate the pH sensor of the sensors 310, the at least one calibration fluid may comprise a 4.01 pH calibration fluid, a 7.01 pH calibration fluid and/or a 10.01 pH calibration fluid. The pH calibration fluids may include components such as water, potassium phthalate and hydrochloric acid in different concentrations to arrive at the different calibrated pHs. Alternatively, to calibrate the ORP sensor of the sensors 310, the calibration fluid may instead comprise a 225 mV ORP calibration fluid. The ORP calibration fluid may include components such as water, potassium ferrocyanide, potassium ferricyanide, potassium chloride in different concentrations to achieve the calibrated ORP reading. In other embodiments, the at least one calibration fluid may comprise a single calibration fluid for calibrating more than one sensor of the sensors 310 together. For example, the at least one calibration fluid may comprise combined 4.01 pH and 225 mV ORP calibration fluid. The combined pH and ORP calibration fluid may include components such as water, potassium phthalate, hydrochloric acid, potassium ferrocyanide, potassium ferricyanide and potassium chloride in different concentrations. In some embodiments, block 462 may also involve providing a user with direction as to an identity of the calibration fluid to be poured the container 208, such as via display on the control interface 160 or on the remote user devices 182.

Operating the water quality monitoring device 140 in the calibration mode 460 may then proceed to block 464, which may involve re-engaging the lower portion 204 (which may be a different lower portion 204A, 204C) with the upper portion 202 while the probe 300 remains in situ relative to the upper portion 202, such that the probe 300 contacts the calibration fluid now held in the container 208. For example, block 464 may involve a user manually manipulating at least one of the body component 272 or the grip component 282 of the lower portion 204 (again, which may be different body and grip components 272A, 272C, 282A and 282C of a different lower portion 204A, 204C) to engage the corresponding upper and lower engagement component 260 and 270 with each other. Once the lower portion 204 is re-engaged with the upper portion 202, the upper and lower portions 202 and 204 may combine to re-form the chamber 206. In other embodiments, such as that shown in FIG. 6D, block 464 may not be performed as the probe 300 may already contact the calibration fluid now held in the container 208F. In such embodiments, the calibration mode 460 may proceed while the lower portion 204F is disengaged from the upper portion 202F and forms the container 208F and while the probe 300 extends into the container 208F and remains in situ relative to the lower portion 204F.

Operating the water quality monitoring device 140 in the calibration mode 460 may then proceed to block 466, which may include directing the probe 300 to obtain the quality measurements from the calibration fluid now held in the re-formed chamber 206 (and/or the container 208F). For example, block 466 may involve causing the controller 122 to transmit the “CALIBRATE” signal to the probe 300 to cause the probe 300 to begin a calibration operation involving the measuring the quality measurements of the calibration fluid with one or more of the sensors 310. As briefly described above, in response to the “CALIBRATE” signal, the probe 300 may operate a particular sensor of sensors 310 for a calibration period of five seconds, 10 seconds, 30 seconds, 60 seconds, etc. The identity of the particular sensor of the sensors 310 to be operated may be based on the identity of the calibration fluid poured into the container 208 at block 462. For example, in response to block 462 directing the user to pour a pH calibration fluid into the container 208, block 466 may involve causing the probe 300 to operate the pH sensor of the sensors 310 for the calibration period. As another example, in response to block 462 directing the user to pour a ORP calibration fluid into the container 208, block 466 may involve causing the probe 300 to operate the ORP sensor of sensors 310 for the calibration period. As another example, in response to block 462 directing the user to pour a combined pH and ORP calibration fluid into the container 208, block 466 may involve causing the probe 300 to a) first operate the pH sensor for a first calibration period and then operate ORP sensor of sensors 310 for a second calibration period and/or b) operate the pH and ORP sensors simultaneous for the calibration period.

Operating the water quality monitoring device 140 in the calibration mode 460 may then proceed to block 468, which may involve transmitting the quality measurements measured by the probe 300 to the controller 122 and/or the remote server 180 for analysis thereof. For example, block 468 may include computer-executable code directing the communication module 150 to transmit the quality measurements to the controller 122 and/or to the remote server 180 along with a tag conveying that the quality measurements are calibration measurements. In some embodiments, block 468 may include computer-executable code directing the communication module 150 to transmit the quality measurements directly to the controller 122 via the communication line(s) 171 as described above or via the network(s) 170 as described above. In other embodiments, block 468 may include computer-executable code directing the communication module 150 to transmit the calibration quality measurements to the remote server 180 via the network(s) 170 as described above.

Operating the water quality monitoring device 140 in the calibration mode 460 may then proceed to block 470, which may involve performing at least one computer-implemented calibration operation of the probe 300 using the controller 122 and/or the remote server 180. The at least one calibration operation is configured to generally adjust the quality measurements measured by the probe 300 for the calibration fluid held in the container 208 to correspond to the known/expected quality measurements of the calibration fluid held in the container 208. For example, if the calibration fluid comprises a 4.01 pH calibration fluid and the quality measurements measured by the probe 300 comprises an electrical potential reading which corresponds to 4.20 pH, at least one of the controller 122 or the remote server 180 may calibrate the reading such that the electrical potential reading corresponds to a 4.01 pH instead. Any suitable method for calibrating observed measurements against expected measurements may be used and will become apparent to those skilled in the art in view of the present disclosure.

Depending on the number of sensors of the sensors 310 of the probe 300, operating the water quality monitoring device 140 in the calibration mode 460 may return to block 462 in some embodiments to undergo another iteration of calibration. For example, a first iteration of calibration may involve calibrating the pH sensor of the sensors 310 at a lower end with the 4.01 pH calibration fluid, while a second iteration of calibration may involve calibrating the same pH sensor at a higher end with the 10.01 pH calibration fluid. As an alternative example, the first iteration of calibration may involve calibrating the pH sensor with one or more pH calibration fluids, while a second iteration of calibration may involve calibrating the ORP sensor of the sensors 310 with a ORP calibration fluid.

For example, a second iteration of operating the water quality monitoring device 140 in the calibration mode 460 may proceed to block 471, which may involve disengaging the lower portion 204 holding the calibration fluid from the upper portion 202 while the probe 300 remains in situ relative to the upper portion 202 as shown in FIG. 5B. For example, block 471 may be similar to block 454 and may involve a user manually manipulating at least one of the body component 272 or the grip component 282 of the lower portion 204 to disengage the upper and lower engagement component 260 and 270 from each other. In some embodiments, such as that shown in FIG. 6D, block 471 may not be performed as the lower portion 204F may not be engaged with the upper portion 202F. The second iteration of the calibration mode 460 may proceed to optional block 472, which may involve discarding the calibration fluid held in the container 208 which was used for the first iteration of the calibration mode 460. In some embodiments, block 472 may involve providing a user with direction as to discard of the calibration fluid, such as via display on the control interface 160 or on the remote user devices 182. The second iteration of the calibration mode 460 may then continue from block 462 as described above to pour another calibration fluid into the container 208, block 464 to re-engage the lower portion 204 with the upper portion 202 to re-form the chamber 206 while the probe 300 remains in situ relative to the upper portion 202 such that the probe contacts the other calibration fluid now held in the chamber 206 (as noted above, in some embodiments, such as that shown in FIG. 6D, block 464 may not be performed as the probe 300 may already contact the calibration fluid held in the container 208F after block 462), block 466 to cause the probe 300 to obtain quality measurements of the other calibration fluid, block 468 to transmit the quality measurements measured by the probe 300 to at least one of the controller 122 or the remote server 180 and then finally block 470 to perform at least one computer-implemented further calibration of the probe 300 using the controller 122 and/or the remote server 180. Alternatively, in embodiments where a single calibration fluid having known properties may be provided for calibrating all desired sensors 310 of the probe 300, only one iteration of one or more of the blocks 462, 464, 466, 468 and 470 of the calibration mode 460 may be performed.

After all desired sensors of the sensors 310 of the probe 300 have been calibrated, operating the water quality monitoring device 140 in the calibration mode 460 may then proceed to block 458 (shown in FIG. 9A) which may involve resuming or otherwise reinitiating flow of the water 104 from the receptacle 102 through the chamber 206. For example, block 458 may involve causing the controller 122 to transmit the “OPEN” or the “x % OPEN” signal to the inlet and outlet valves 214 and 224, the combined at least one valve, and the inlet and outlet valve locks 216 and 226 or the combined at least one valve lock to direct the inlet and outlet valves 214 and 224 to open (or partially open) the respective inlet and outlet ports 210 and 220 or the combined at least one valve and to direct the inlet and outlet valve locks 216 and 226 or the combined at least one valve lock to lock the respective inlet and outlet ports 210 and 220 in the open position (or the partially open position). This allows the water 104 from the receptacle 102 to flow into the chamber 206 via the inlet port 210, through the chamber 206, and out of the chamber 206 via the outlet port 220 to return to the receptacle 102. Block 458 may be similar to the block 404 of the in the water quality monitoring mode 400 shown in FIG. 8, and the water quality monitoring device 140 may resume operating in the water quality monitoring mode 400 after block 458 of the maintenance modes 450.

In some embodiments, block 458 may be performed after block 470 while the calibration fluid remains in the chamber 206. In such embodiments, the calibration fluid may be flushed into the receptacle 102 by the flow of the water 104 through the chamber 206. Such embodiments may be used where the calibration fluid does not contain chemical components that could be harmful to components of the bathing unit system 100 and/or to users of the bathing unit system 100. However, in some embodiments, block 458 may be performed after block 472 after the calibration fluid has been discarded from the container 208. In such embodiments, operating the water quality monitoring device 140 in the calibration mode 460 may further involve proceeding to block 474 from block 472, which may involve re-engaging the lower portion 204 holding nothing in the container 208 (or holding the water 104 from the bathing unit system 100 in the container 208) with the upper portion 202 while the probe 300 continues to remain in situ relative to the upper portion 202 (or while the probe 300 continues to remain in situ relative to the lower portion 204F). Block 474 may be similar to block 464 and may involve a user manually manipulating at least one of the body component 272 or the grip component 282 of the lower portion 204 to engage the corresponding upper and lower engagement component 260 and 270 with each other. Performing block 458 after blocks 472 and 474 may prevent any calibration fluid from being flushed into the receptacle 102 by the flow of the water 104 through the chamber 206 and may be desirable when the calibration fluid does include chemical components that can be harmful to components of the bathing unit system 100 and/or to users of the bathing unit system 100.

### Storage Mode 480

Still referring to FIGS. 9A and 9B, in some embodiments, operating the water quality monitoring device 140 in the storage mode 480 may also begin at the optional block 457, which may involve switching the lower portion 204 disengaged from the upper portion 202 for another lower portion 204. This other lower portion may comprise the lower portion 204A having the smaller container volume (shown in FIG. 6A) or the lower portion 204C having the smaller container volume (shown in FIG. 6C). Similar to the calibration mode 460 described above, switching the lower portion 204 for another lower portion 204A, 204C having the smaller container volume can reduce the volume of storage fluid required to submerge the probe 300 as described below. Use of a smaller volume of storage fluid may be desirable as storage fluid may also be costly.

Operating the water quality monitoring device 140 in the storage mode 480 may then proceed to block 482, which may involve pouring the storage fluid into the container 208 formed by the lower portion 204 (which may be a different container formed by a different lower portion 204A, 204C) when it is disengaged from the upper portion 202. The storage fluid may be used for storing the probe when the bathing unit system 100 is not in use. The storage fluid may be a fluid having a chemical composition which reduces degradation of the probe 300, including of the probe body 302 and of the sensors 310. As a more specific example, the storage fluid may be a fluid having a particular chemical composition which reduces degradation of the ORP sensor of the sensors 310. For example, in some embodiments, the storage fluid may comprise water and potassium chloride (KCl). In specific practical embodiments, the storage fluid may comprise water, potassium phthalate and potassium chloride.

Operating the water quality monitoring device 140 in the storage mode 480 may then proceed to block 483, which may involve re-engaging the lower portion 204 (which may be a different lower portion 204A, 204C) with the upper portion 202 while the probe 300 remains in situ relative to the upper portion 202, such that the probe 300 contacts the storage fluid now held in the container 208. In some embodiments, such as that shown in FIG. 6D, the probe 300 may already contact the calibration fluid held in the container 208F after block 482. In such embodiments, block 483 may involve re-engaging the lower portion 204F to the upper portion 202F while the probe 300 extends into the container 208F and remains in situ relative to the lower portion 204F. For example, block 483 may be similar to blocks 464 and 474 described above, and may involve a user manually manipulating at least one of the body component 272 or the grip component 282 of the lower portion 204 (again, which may be different body and grip components 272A, 272C, 282A and 282C of a different lower portion 204A, 204C) to engage the corresponding upper and lower engagement components 260 and 270 with each other. Once the lower portion 204 is re-engaged with the upper portion 202, the upper and lower portions 202, 204 may combine to re-form the chamber 206. The water quality monitoring device 140 may be maintained at block 482 for an extended period of time to store the probe 300, such as when the bathing unit system 100 is not in use (e.g., during months in winter or other non-use periods).

Operating the water quality monitoring device 140 in the storage mode 480 may then proceed to block 484, which may involve disengaging the lower portion 204 holding the storage fluid from the upper portion 202 while the probe 300 continues to remain in situ relative to the upper portion 202. In some embodiments, such as that shown in FIG. 6D, block 484 may involve disengaging the lower portion 204F from the upper portion 202F while the probe 300 remains in situ relative to the lower portion 204F. For example, block 484 may be similar to blocks 454, and 471 described above, and may also involve a user manually manipulating at least one of the body component 272 or the grip component 282 of the lower portion 204 to disengage the upper and lower engagement components 260 and 270 from each other. Operating the water quality monitoring device 140 in the storage mode 480 may then proceed to block 486, which involves discarding the storage fluid held in the container 208. For example, block 486 may be similar to block 472, and may involve providing a user with direction as to discard of the storage fluid, such as via display on the control interface 160 or on the remote user devices 182. Operating the water quality monitoring device 140 in the storage mode 480 may then proceed to block 474 as described above to re-engage the lower portion 204 holding nothing in the container 208 (or holding the water 104 from the bathing unit system 100 in the container 208) with the upper portion 202 while the probe 300 continues to remain in situ relative to the upper portion 202 (or while the probe 300 continues to remain in situ relative to the lower portion 204F) and then to block 458 as described above to resume flow of the water 104 from the receptacle 102 through the chamber 206.

In some embodiments, block 458 may be performed after block 474 such that there is no storage fluid remaining in the chamber 206. Such embodiments may prevent any storage fluid from being flushed into the receptacle 102 by the flow of the water 104 through the chamber 206 and may be desirable when the storage fluid includes chemical components that can be harmful to components of the bathing unit system 100 and/or to users of the bathing unit system 100. However, in other embodiments, block 458 may be performed directly after block 483 while the storage fluid remains in the chamber 206. In such embodiments, the storage fluid may be flushed into the receptacle 102 by the flow of the water 104 through the chamber 206 and may be used where the storage fluid does not contain chemical components that could be harmful to components of the bathing unit system 100 and/or to users of the bathing unit system 100.

### Cleaning Mode 500

Still referring to FIGS. 9A and 9B, operating the water quality monitoring device 140 in the cleaning mode 500 may begin at block 502, which may involve cleaning the probe 300 while the probe is exposed and remains in situ relative to the upper portion 202 (or while the probe 300 extends into the container 208F and remains in situ relative to the lower portion 204F). For example, in some embodiments, block 502 may involve brushing the probe 300 or the sensors 310 with a brush or wiping the probe 300 or the sensors 310 with a cloth. In other embodiments, block 502 may involve soaking the probe 300 with a cleaning fluid and then wiping off or rinsing the cleaning fluid. In such embodiments, the cleaning fluid may comprise water and hydrogen chloride. In embodiments involving soaking the probe 300 with the cleaning solution, block 502 may involve steps similar to blocks 457, 482, 483 and 486 of the storage mode 480. For example, block 502 may involve switching the disengaged lower portion 204 for another lower portion 204A, 204C having the smaller container volume (similar to block 457); and pouring the cleaning fluid into the container 208 formed by the lower portion 204, 204A, 204C (similar to block 482). Block 502 may further involve re-engaging the lower portion 204, 204A, 204C, with the container 208 now holding the cleaning fluid, with the upper portion 202 such that the probe 300 contacts and/or is submerged the cleaning fluid (similar to block 483) and holding the probe 300 submerged in the cleaning fluid for a cleaning period of time (e.g., anywhere between approximately 5 minutes and approximately 30 minutes), and then disengaging the lower portion 204, 204A, 204C holding the cleaning fluid from the upper portion 202 after the cleaning period of time (similar to block 484). In other embodiments, such as that shown in FIG. 6D, block 502 may not involve switching the lower portion 204F for another lower portion 204 as the probe 300 may be retained by the lower portion 204F and may also not involve further re-engaging and then re-disengaging the lower portion 204F with the upper portion 202F when the cleaning fluid is received in the container as the probe 300 may already contact the cleaning fluid held in the container 208F. In such embodiments, the cleaning mode 500 may proceed while the lower portion 204F is disengaged from the upper portion 202F and forms the container 208F and while the probe 300 extends into the container 208F and remains in situ relative to the lower portion 204F. Block 502 may further involve discarding the cleaning fluid from the container 208, 208F (similar to block 486).

Operating the water quality monitoring device 140 in the cleaning mode 500 may then continue from block 474 as described above to re-engage the lower portion 204 holding nothing in the container 208, 208F (or holding the water 104 from the bathing unit system 100 in the container 208, 208F) with the upper portion 202 while the probe 300 continues to remain in situ relative to the upper portion 202 (or while the probe 300 continues to remain in situ relative to the lower portion 204F) and then to block 458 as described above to resume flow of the water 104 from the receptacle 102 through the chamber 206. In some embodiments, block 458 may be performed when there is no cleaning fluid remaining in the chamber 206 (e.g., after the cleaning fluid has been discarded from the container 208 or when block 502 merely involves brushing or wiping the probe 300). Such embodiments may prevent any cleaning fluid from being flushed into the receptacle 102 by the flow of the water 104 through the chamber 206 and may be desirable when the cleaning fluid includes chemical components that can be harmful components of the bathing unit system 100 and/or to users of the bathing unit system 100. However, in other embodiments, block 458 may be performed when there is cleaning fluid remaining in the chamber 206 (e.g., where the lower portion 204 is not further disengaged to discard the cleaning fluid from the container 208). In such embodiments, the cleaning fluid may be flushed into the receptacle 102 by the flow of the water 104 through the chamber 206 and may be used where the cleaning fluid does not contain chemical components that could be harmful to components of the bathing unit system 100 and/or to users of the bathing unit system 100.

### Conclusion

The person skill in the art will appreciate that many variations to the embodiments described in the present document art possible and will become apparent from a reading of the present document concurrently with the figures.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, the terms “around”, “about”, “substantially” or “approximately” shall generally mean within the error margin generally accepted in the art. Hence, numerical quantities given herein generally include such error margin such that the terms “around”, “about” “substantially” or “approximately” can be inferred if not expressly stated. For greater clarity, unless otherwise explicitly stated, the terms “around”, “about”, “substantially” and “approximately” means a proportion of at least about 60%, or at least about 70% or at least about 80%, or at least about 90%, at least about 95%, at least about 97% or at least about 99% or more, or any integer between 70% and 100%.

Note that the expression “at least one of A or B”, as used herein, is interchangeable with the expression “A and/or B”. It refers to a list in which you may select A or B or both A and B. Similarly, “at least one of A, B, or C”, as used herein, is interchangeable with “A and/or B and/or C” or “A, B, and/or C”. It refers to a list in which you may select: A or B or C, or both A and B, or both A and C, or both B and C, or all of A, B and C. The same principle applies for longer lists having a same format.

Although various embodiments of the invention have been described and illustrated, it will be apparent to those skilled in the art in light of the present description that numerous modifications and variations can be made. The scope of the invention is defined more particularly in the appended claims.

1. An inline water quality monitoring device for use with a bathing unit system, the bathing unit system including a receptacle for holding water and a circulation system for removing water from and returning water to the receptacle, the inline water quality monitoring device comprising:
a) a housing comprising an upper portion and a lower portion which forms a chamber, wherein the housing is configured to be fluidly coupled with the circulation system to allow water from the receptacle to flow through the chamber, and wherein the lower portion is releasably engaged with the upper portion and forms a container for holding fluid when disengaged from the upper portion; and
b) a probe coupled to the upper portion of the housing and extending into the chamber, wherein the probe is configured to obtain quality measurements of the water from the receptacle which flows though the chamber and wherein, when the lower portion is disengaged from the upper portion, the probe is exposed and remains in situ relative to the upper portion.

2. The inline water quality monitoring device of claim 1, wherein the lower portion is releasably engaged with the upper portion by being at least one of threadedly releasably engaged with the upper portion, magnetically releasably engaged with the upper portion, frictionally releasably engaged with the upper portion, or rotationally releasably engaged with the upper portion.

3. The inline water quality monitoring device of claim 1, wherein the lower portion comprises a grip component on an outer surface of the lower portion.

4. The inline water quality monitoring device of claim 3, wherein the grip component comprises a plurality of vane grips outwardly extending from the outer surface of the lower portion.

5. The inline water quality monitoring device of claim 1, wherein the container formed by the lower portion has at least one of a U-shaped cross-section or a V-shaped cross-section.

6. The inline water quality monitoring device of claim 1, wherein the container formed by the lower portion is at least one of transparent or translucent.

7. The inline water quality monitoring device of claim 1, wherein, when used in a calibration mode, the container holds calibration fluid for calibrating the probe.

8. The inline water quality monitoring device of claim 7, wherein the calibration fluid comprises a fluid having known quality measurements to calibrate the quality measurements obtained by the probe.

9. The inline water quality monitoring device of claim 8, wherein the lower portion with the calibration fluid held in the container is configured to be releasably re-engaged with the upper portion while the probe remains in situ relative to the upper portion such that the probe contacts the calibration fluid held in the container.

10. The inline water quality monitoring device of claim 1, wherein, when used in a storage mode, the container holds storage fluid for storing the probe.

11. The inline water quality monitoring device of claim 10, wherein the storage fluid comprises a fluid having a chemical composition that reduces degradation of the probe.

12. The inline water quality monitoring device of claim 11, wherein the storage fluid comprises a potassium chloride (KCl) solution.

13. The inline water quality monitoring device of claim 11, wherein the lower portion with the storage fluid held in the container is configured to be releasably re-engaged with the upper portion while the probe remains in situ relative to the upper portion such that the probe contacts the storage fluid held in the container.

14. The inline water quality monitoring device of claim 1, wherein, when used in a cleaning mode, the lower portion is disengaged from the upper portion to allow a user to access the probe while the probe is exposed and remains in situ relative to the upper portion.

15. The inline water quality monitoring device of claim 1, wherein the lower portion is a first lower portion defining a first container, and the first lower portion is configured to be disengaged from the upper portion to allow a second lower portion defining a second container different from the first container to be releasably engaged with the upper portion.

16. The inline water quality monitoring device of claim 15, wherein the first container defined by the first lower portion has a first volume and the second container defined by the second lower portion has a second volume smaller than the first volume.

17. The inline water quality monitoring device of claim 16, wherein the second container holds calibration fluid for calibrating the probe when the inline water quality monitoring device is used in a calibration mode.

18. The inline water quality monitoring device of claim 16, wherein the second container holds storage fluid for storing the probe when the inline water quality monitoring device is used in a storage mode.

19. The inline water quality monitoring device of claim 1, wherein the housing comprises an inlet port and an outlet port to fluidly couple the housing with the circulation system, and wherein the chamber is positioned between the inlet port and the outlet port such that at least part of the water from the receptacle enters the chamber through the inlet port and exits the chamber through the outlet port.

20. The inline water quality monitoring device of claim 19, wherein the inlet port extends upwardly from the chamber and the outlet port extends downwardly from the chamber.

21. The inline water quality monitoring device of claim 19, further comprises at least one valve configured to open and close at least one of the inlet port or the outlet port, wherein the at least one valve comprises:
a) at least one inlet valve configured to open and close the inlet port and at least one outlet valve configured to open and close the outlet port; or
b) a combined at least one valve configured to open and close both the outlet port and the inlet port.

22. The inline water quality monitoring device of claim 21, wherein the at least one valve is configured to be:
a) locked in a closed position when the inline water quality monitoring device is used in at least one of a calibration mode, storage mode or a cleaning mode; or
b) locked in an open position when the inline water quality monitoring device is used in a monitoring mode.

23. The inline water quality monitoring device of claim 1, further comprising a support component extending from the upper portion of the housing, wherein the support component is configured to fixedly coupled the upper portion to the bathing unit system such that, when the lower portion is disengaged from the upper portion, the probe remains in situ relative to the bathing unit system.

24. The inline water quality monitoring device of claim 1, wherein the probe comprises at least one of a pH sensor, an oxidation-reduction sensor (ORP sensor), and a temperature sensor.

25. A method for in situ maintenance of an inline water quality monitoring device, the inline water quality monitoring device comprising A) a housing comprising an upper portion and a lower portion defining a chamber, wherein the housing is configured to be fluidly coupled with a circulation system of a bathing unit system to allow water from a receptacle of the bathing unit system to flow through the chamber, and wherein the lower portion is releasably engaged with the upper portion and forms a container for holding fluid when disengaged from the upper portion and B) a probe extending into the chamber through the upper portion and configured to measure water quality measurements of the water from the receptacle which flows though the chamber, the method comprising:
a) ceasing flow of the water from the receptacle through the chamber of the inline water quality monitoring device;
b) disengaging the lower portion from the upper portion to expose the probe; and
c) performing at least one maintenance operation on the probe while the probe remains in situ relative to the upper portion.

26. The method of claim 25, wherein the lower portion comprises a grip component on an outer surface of the lower portion, wherein disengaging the lower portion from the upper portion comprises:
a) disengaging the lower portion from the upper portion by manipulating the lower portion via the grip component.

27. The method of claim 26, wherein performing the at least one maintenance operation comprises operating the inline water quality monitoring device in one of:
a) a calibration mode;
b) a cleaning mode; or
c) a storage mode.

28. The method of claim 27, wherein operating the inline water quality monitoring device in the calibration mode comprises:
a) pouring calibration fluid having known quality measurements into the container formed by the lower portion; and
b) re-engaging the lower portion with the upper portion while the probe remains in situ relative to the upper portion such that the probe contacts the calibration fluid held in the container.

29. The method of claim 28, wherein operating the inline water quality monitoring device in the calibration mode further comprises:
a) using the probe to obtain quality measurements corresponding to the calibration fluid;
b) transmitting the obtained quality measurements to at least one processor associated with the bathing unit system; and
c) performing, with the at least one processor, a computer-implemented calibration operation including processing the quality measurements obtained by the probe and the known quality measurements of the calibration fluid.

30. The method of claim 29, wherein at least part of the computer-implemented calibration operation is performed on a remote server physically separate from the bathing unit system.

31. The method of claim 27, wherein operating the inline water quality monitoring device in the storage mode comprises:
a) pouring storage fluid having a chemical composition which reduces degradation of the probe into the container formed by the lower portion; and
b) re-engaging the lower portion with the upper portion while the probe remains in situ relative to the upper portion such that the probe contacts the storage fluid held by the container.

32. The method of claim 31, wherein operating the inline water quality monitoring device in the cleaning mode comprises:
a) cleaning the probe while the probe is exposed and remains in situ relative to the upper portion;
b) re-engaging the lower portion with the upper portion; and
c) reinitiating the flow of the water from the receptacle to the chamber such that the cleaned probe contacts the water from the receptacle flowing through the chamber.

33. The method of claim 25, wherein the housing comprises an inlet port and an outlet port to fluidly couple the housing with the circulation system, and wherein the chamber is positioned between the inlet port and the outlet port such that at least part of the water from the receptacle enters the chamber through the inlet port and exits the container through the outlet port, wherein ceasing flow of the water from the receptacle into the container comprises:
a) closing at least one valve associated with at least one of the inlet port and the outlet port, wherein the at least one valve comprises:
i) at least one inlet valve configured to open and close the inlet port and at least one outlet valve configured to open and close the outlet port; or
ii) a combined at least one valve configured to open and close both the outlet port and the inlet port.

34. The method of claim 33, further comprising:
a) locking the at least one valve in a closed position when the inline water quality monitoring device is used in at least one of a calibration mode, storage mode or a cleaning mode; and
b) locking the at least one valve in an open position when the inline water quality monitoring device is used in a monitoring mode.

35. The method of claim 34, wherein the lower portion is a first lower portion defining a first container having a first volume and wherein performing the at least one maintenance operation comprises:
a) releasably engaging a second lower portion with the upper portion while the probe remains in situ relative to the upper portion, wherein the second lower portion defines a second container having a second volume different from the first volume.

36. The method of claim 35, further comprising:
a) pouring at least one of a calibration fluid or a storage fluid into the second container defined by the second lower portion prior to releasably engaging the second lower portion with the upper portion.

37. The method of claim 25, wherein the method further comprises:
a) fixedly coupling the upper portion of the housing to the bathing unit system such that performing the at least one maintenance operation on the probe while the probe remains in situ relative to the upper portion comprises performing the at least one maintenance operation on the probe while the probe remains in situ relative to the bathing unit system.

38. An inline water quality monitoring device for use with a bathing unit system, the bathing unit system including a receptacle for holding water and a circulation system for removing water from and returning water to the receptacle, the inline water quality monitoring device comprising:
a) a housing comprising an upper portion and a lower portion which forms a chamber, wherein the housing is configured to be fluidly coupled with the circulation system to allow water from the receptacle to flow through the chamber, and wherein the lower portion is releasably engaged with the upper portion and forms a container for holding fluid when disengaged from the upper portion; and
b) a probe coupled to the housing and extending into the chamber, wherein the probe is configured to obtain quality measurements of the water from the receptacle which flows though the chamber and wherein, when the lower portion is disengaged from the upper portion, the probe remains in situ relative to the housing.

39. The inline water quality monitoring device of claim 38, wherein the probe is coupled to the upper portion of the housing and, when the lower portion is disengaged from the upper portion, the probe:
a) is exposed; and
b) remains in situ relative to the upper portion.

40. The inline water quality monitoring device of claim 38, wherein the probe is coupled to the lower portion of the housing and, when the lower portion is disengaged from the upper portion, the probe:
a) extends into the container; and
b) remains in situ relative to the lower portion.

41. A method for in situ maintenance of an inline water quality monitoring device, the inline water quality monitoring device comprising A) a housing comprising an upper portion and a lower portion defining a chamber, wherein the housing is configured to be fluidly coupled with a circulation system of a bathing unit system to allow water from a receptacle of the bathing unit system to flow through the chamber, and wherein the lower portion is releasably engaged with the upper portion and forms a container for holding fluid when disengaged from the upper portion and B) a probe coupled to the housing and extending into the chamber and configured to measure water quality measurements of the water from the receptacle which flows though the chamber, the method comprising:
a) ceasing flow of the water from the receptacle through the chamber of the inline water quality monitoring device;
b) disengaging the lower portion from the upper portion; and
c) performing at least one maintenance operation on the probe while the probe remains in situ relative to the housing.

42. The method of claim 41, wherein the probe is coupled to the upper portion of the housing, wherein disengaging the lower portion from the upper portion exposes the probe while the probe remains in situ relative to the upper portion, and wherein performing the at least one maintenance operation on the probe comprises performing at least one maintenance operation on the probe while the probe is exposed and remains in situ relative to the upper portion.

43. The method of claim 41, wherein the probe is coupled to the lower portion of the housing and extends into the container, wherein the probe extends into the container when the lower portion is disengaged from the upper portion, and wherein performing the at least one maintenance operation on the probe comprises performing at least one maintenance operation on the probe while the probe extends into the container and remains in situ relative to the lower portion.
